(19) United States
(12) Patent Application Publication
(10) Pub. No.: US 2010/0190737 A1
(43) Pub. Date: Jul. 29, 2010

(54) COMPOSITIONS COMPRISING INOSINE AND OROTIC ACID AND METHODS OF USE THEREOF FOR THE TREATMENT OF CERTAIN HEART CONDITIONS AND ENHANCEMENT OF WORK CAPACITY

(75) Inventors: Yevsey Belenky, Brooklyn, NY (US); Jeffrey Shapiro, New Rochelle, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: NUTRITIONAL RESEARCH GROUP LLC, Bohemia, NY (US)

(21) Appl. No.: 12/359,366

(22) Filed: Jan. 26, 2009

(51) Int. Cl. A61K 31/7076; A61P 9/00

(52) U.S. Cl. 514/46

(57) ABSTRACT

The invention comprises a composition of inosine and orotic acid or a salt thereof, and its methods of use in treating, maintaining and enhancing the health of the heart, and specifically the integrity of the myocardium. The effective combination of inosine and orotic acid/orotate effectively improves various medical parameters that are widely used to assess cardiac function and structure. These include EKG and VCG recordings, quantitative assessments of work capacity and athletic performance, clinically relevant observations, and direct biochemical, histological and ultrastructural analyses. The observations and controlled studies in mice, rats, rabbits, cardiology patients and high performance human athletes (cyclists) consistently support the effectiveness of the combination of inosine and orotic acid/orotate in both preventing and reversing damage to the myocardium resulting from physical stress.

### FIELD OF THE INVENTION

The invention relates to the fields of medicine, pharmaceuticals and nutraceuticals for improving cardiac function and overall health.

### BACKGROUND OF THE INVENTION

Cardiovascular diseases (CVD) are statistically the number one cause of death in the world. The American Heart Association (AHA) estimates that more than 80 million people in the United States have had, or are at risk of experiencing one or more forms of CVD, including heart failure (HF), myocardial infarction (MI) and myocardiodystrophy. An additional 16 million people experience some degree of coronary heart disease (CHD). It is estimated that there are 900,000 deaths from cardiovascular diseases annually with approximately 600,000 of which are attributed to CHD and myocardial infarction (MI/heart attack). CHD can have various causes, from acute trauma to slow progressing long term damage to the myocardium. The long term damage that accumulates over time can, e.g., be due to emotional and mechanical stresses to the heart. Mechanical stresses arise due to long term conditions and habits, e.g., obesity, poor diet leading to the constriction of arteries and increases in plaque deposits and an overly sedentary lifestyle. However, it may appear ironic that even activities that are commonly viewed as being beneficial to one's health, e.g., exercise and other forms of physical work and recreation, may ultimately provide an additional basis of CHD or cardiac insufficiency when the individual over-trains. Over-training or over-indulging in athletics or physically demanding worl of any kind occurs when an individual exceeds his or her limits of enduring physical stress. How much physical stress can be safely experienced is determined by the individual's current physical condition generally, and the health of the individual's heart specifically.

Initially an over-stressed heart condition usually results in positive compensatory morphological and biochemical changes that generally enhance health. However, if these compensatory positive changes are insufficient to relieve the extent of the over-stressed heart, the myocardium will continue to work at a stressed level and may begin to develop negative, i.e., pathological, changes from the remodeling of the heart or even a detertoration, i.e., dystrophy, of the myocardiurn, resulting in a damaged heart.

During chronic cardiac overload, which may arise from a cardiovascular disease condition such as hypertension, ischemic heart disease, valve disorders, excessive exercise or obesity, the heart compensates by developing concentric (“good”) heart hypertrophy. Concentric heart hypertrophy is a beneficial condition wherein the cardiac overload results in an increase in the mass of myocardial tissue, which is manifested as a thickening of the ventricular muscle, but without any substantial enlargement of the heart cavities. However, if the concentric heart hypertrophy is not adequate to ameliorate the overstrained condition, the heart will continue attempting to compensate for the overstrain and may thereafter develop into an eccentric heart hypertrophy (EHH) which is a negative or “bad” condition characterized by the dilation of the ventricular chamber and comprises a distinguishing characteristic of heart failure.

Concentric heart hypertrophy enhances the heart's productivity and, specifically, the left ventricle's pumping action by increasing the heart weight and myocyte size, leading to a reduced mechanical load on individual sarcomeres. If the level of concentric heart hypertrophy is sufficient to provide the necessary amount of blood and oxygen supply to the organs, no additional change in the heart's anatomy ensues, and the heart remains in the concentric heart hypertrophy condition. However, if the concentric heart hypertrophy is not sufficient to provide an adequate blood and oxygen supply to the other organs, the heart will still function in an overloaded condition, and the development of eccentric heart hypertrophy is likely.

### SUMMARY OF THE INVENTION

In view of the foregoing discussion, it is clear that there is a need in the medical arts to provide compositions capable of addressing various prevalent forms of cardiac disease. There is a further need to improve the cardiac health of individuals that perform physical work and may not be aware of the long term consequences of the physical stresses placed on the heart. There is an even her need in the art to provide assistance to athletes, whether they are performing on a competitive level or recreational level, with means of improving their cardiac health and enhance their work capacity without the potentially deleterious effects of prolonged stress to the heart that can accompany training and especially over training, as well as other forms of physical activity, including physical work and athletics.

The embodiments of the composition of the present invention and methods of use thereof, relate to non-toxic compositions comprising the combination of inosine or pharmaceutically acceptable salts thereof or esters thereof, including phosphate esters and orotic acid or acylating derivatives thereof or pharmaceutically acceptable salts thereof for use as pharmaceutical or nutraceutical supplements. The composition comprises a combination of active ingredients that together act to ameliorate, maintain and even prevent the progression of pathological medical conditions resulting from over stressing the heart. In an embodiment of the present invention, the inosine and/or esters thereof or salts thereof and orotic acid and/or any acylating derivatives thereof or salt thereof are present in the composition in synergistic effective amounts, the inosine or salts thereof or esters thereof as described hereinabove and orotic acid or acylating derivative of orotic acid or the pharmaceutically acceptable salts of orotic acid being present in an amount sufficient to treat the medical condition of a mammalian heart. In an embodiment, the composition excludes a lysine salt of orotic acid. It is further desired to provide a combination of compounds that can treat or prevent one or more medical conditions of a mammalian heart, such as heart failure (HF), myocardial infarction, myocardiodystrophy, arrhythmias and tissue damage due to the physical stress of overtraining, i.e., myocardiodystrophy, heart failure and the like. An additional benefit of such pharmaceutical or nutraceutical supplements is enhancing work capacity (physical performance) without inducing eccentric heart hypertrophy, i.e., a pathological over-dilation of the ventricle.

Therefore, one aspect of the present invention encompasses a combination comprising an amount of inosine and orotic acid, or acylating derivative thereof or one or more inorganic salts thereof, effective to enhance the health of a mammalian heart. The Weight ratio of inosine or esters thereof as described hereinabove or salt thereof to orotic acid or acylating derivative thereof or salt thereof or an orotate salt thereof or esters in the administered combination may range from about approximately 1:10 to about 10:1.

Reference to inorganic salts of orotic acid is meant to signify that the orotate anion forms an ionic bond with a cation. The cation, in an embodiment, is a spectator ion, that is, does not materially affect if at all the medical condition of the heart relative to the combination of compounds described herein. The cation is in an embodiment an inorganic ion, i.e., comprised of atoms other than carbon atoms or contains no more than one or two carbon atoms, except for ammonium cation, where the substituents to the central nitrogen atom may independently be lower alkyl, as defined herein or hydrogen. In another embodiment, the cation is an alkali metal, an alkaline earth metal, or a metal of Group VIIb, Group VIIIb, Group Ib or Group IIb. The cation is, in another embodiment, a monovalent or divalent metal or trivalent metal. Persons of ordinary skill in the art will appreciate that in formulating the combination of inosine and/or the various esters or salts and orotic acid and/or the various esters or salts, the total orotic acid/orotate component may comprise one or more salts of orotic acid, as long as the weight ratio of inosine to the orotic acid/orotate component falls within the specified inosine to orotate ratio. Thus, the inorganic orotate salts may have as the inorganic cation one or more of lithium, potassium, sodium, calcium, iron (e.g., ferrous or ferric), magnesium, manganese or zinc, or other pharmaceutically suitable inorganic cations.

An additional aspect of the invention is to provide a composition that is relatively simple to administer. Therefore, one aspect of the invention provides pharmaceutical and nutraceutical formulations suitable for oral, enteric, buccal, intravenous or subcutaneous administration. Accordingly, the pharmaceutical and nutraceutical compositions may include, but are not limited to aqueous or alcoholic solutions, caplets, tablets, gelcaps, capsules or powders and the like. Other formulations can also be prepared according to alternative routes of administration, e.g., parenteral, rectal, transdermal, lingual, intralingual, and sublingual. In an embodiment, the composition may be added to a beverage, such as bottled water, flavored water, or soda, juice, coffee, milk, and the like.

This invention further provides for methods and regimens for administering a composition comprising an amount of inosine or an ester thereof or pharmaceutically acceptable salt thereof and an amount of orotic acid, or acylating derivative or a pharmaceutically acceptable salt thereof. In one embodiment of the invention, the amounts of inosine or ester or pharmaceutically acceptable salt thereof, and orotic acid or acylating derivative or pharmaceutically acceptable are within a specified range of weight ratios, and the dosages are such that the composition has a markedly compound effect relative to the individual components, for example. In another embodiment, the combination has synergistic effects. This composition may be used in methods for treating or preventing certain cardiovascular conditions, in animals and humans, including Heart Failure (HF), myocardial infarction (MI), myocardiodystrophy, arrhythmias and in methods for enhancing physical endurance or work capacity without development of HF or myocardiodystrophy.

Accordingly, one aspect of the invention provides for a method for treating a medical heart condition in a subject, the method comprising, administering to the subject in need thereof an amount of a combination of inosine or ester or pharmaceutically acceptable salt thereof and orotic acid, or acylating derivative thereof or one or more inorganic salts of orotic acid effective to treat cardiac insufficiency. The aforesaid combination may be administered as a pharmaceutical, a nutraceutical, or other kind of nutrient or dietary supplement. In another aspect of the present invention, the method provides for preventing a medical condition in a subject, the method comprising administering to the subject in need thereof, an amount of a combination of inosine or ester or pharmaceutically acceptable salt thereof and orotic acid, or acylating derivative thereof or salt of orotic acid effective to treat cardiac insufficiency.

The method of the present invention has wide applicability as disclosed herein. Accordingly, an additional aspect of the invention is to provide a method for specifically treating or preventing cardiac insufficiency caused by heart failure, a myocardial infarction, arrhythmia, cardiomyopathy or myocardiodystrophy by administering the combination of inosine or pharmaceutically acceptable salt thereof or ester thereof and orotic acid or acylating derivative thereof or a pharmaceutically acceptable salt thereof in effectively cardioprotective amounts, said inosine or pharmaceuticaliy acceptable salt or ester thereof, and orotic acid or acylating derivative thereof or pharmaceutically acceptable salt thereof being present in amounts effective to treat cardiac insufficiencies.

In additional embodiments, the cardioprotective amounts of inosine or pharmaceutically acceptable salt or ester thereof, and orotic acid or acylating derivative thereof or pharmaceutically acceptable salt thereof, are combined in proportions that synergistically provide cardioprotective effects.

In view of the centrality of the heart to basic health, mobility and personal independence, it is an additional aspect of the invention to provide a method of increasing a subject's cardiac work capacity, the method comprising administering to a subject, an effective amount of a combination of inosine or ester or pharmaceutically acceptable salt thereof and orotic acid or ester or acylating derivative or pharmaceutically acceptable salts thereof, effective to enhance the work capacity performed by the subject. In another embodiment, the amount administered is a synergistic effective amount.

### BRIEF DESCRIPTION OF THE DRAWINGS

FIG. 1 presents the EKG recording of a 42 year old female who exhibited a ventricular premature beat arrhythmia prior to treatment with the composition of the present invention.

FIG. 2 shows the EKG of the individual having the arrhythmia recorded in FIG. 1, after 30 days of taking four capsules daily of inosine plus magnesium orotate (400 mg/300 mg, respectively, per capsule).

FIG. 3 shows an EKG recording of a 74 year old male with arrhythmia prior to treatment with the composition of the present invention.

FIG. 4 shows an EKG recording from the 74 year old man with arrhythmia in FIG. 3 after taking six capsules daily for 12 days of inosine plus magnesium orotate (400 mg/300 mg per capsule, respectively) showing that the arrhythmias were resolved. (See FIGS. 3 and 4, EKG graphs).

FIG. 5 provides representative macroscopic views of (A) the heart of intact rat, (B) the heart demonstrating concentric hypertrophy (rat with experimentally induced aortic stenosis, and receiving an inosine supplement), and (C) an example of a heart having eccentric hypertrophy (rat with experimentally induced aortic stenosis, but not treatment with inosine).

FIG. 6 shows light micrographs of haemotoxylin-eosin stained tissue sections (Mag.10×20) of the myocardium of (A) a rat with experimentally induced aortic stenosis Group II, and (B) a rat with experimentally induced aortic stenosis, and receiving iiosine (Group III) or inosine plus orotate (Group IV). The rat in Group II showed evidence of numerous hemorrhages and the formation of acicular (i.e., elongated needle shape) cavitities. The fibers of myocardium in panel (A) appear to be thinner and disrupted, as would be expected from resorption or atrophy of the myocardium. In contrast however, is the normal appearance of the myocardiurn of rats from Groups III and IV, panel (A), which had aortic stenosis but were also treated with inosine only (Group III), or the combination of inosine and orotate (Group IV), respectively.

FIG. 7 shows the ultrastructure of the left ventricular myocardium in a state of (A) eccentric hypertrophy, (Group II), and (B) concentric hypertrophy (Groups III and IV) as described in the description of FIG. 6. Panel (A) shows that the myocardium of rats in Group II having aortic stenosis without supplements, show enlarged mitochondria and distended sarcoplasmic reticulum cistemae (SPR). Panel (B) shows that the myocardium of rats in Group III and Group IV having aortic stenosis but were also treated with inosine (Group III), or the combination of inosine and orotate (Group IV), respectively, show normal sized mitochondria.

FIG. 8 shows a slide of untreated rabbit myocardium on day 14 after coronary artery ligation. Light microscopy (10×10). Image shows the replacement of necrotic muscle fibers within the zone of infarction by newly elaborated loose connective tissue. The lack of relatively uniform striation indicates a lack scar tissue.

FIG. 9 shows a slide of rabbit myocardium after treatment/administration of inosine and orotic acid admixture for 14 days after coronary ligation. A cicatrix (new tissue/scarring), predominantly made of collagen fibers and few cellular elements, is formed in the zone of infarction.

FIG. 10 shows a slide of untreated control group rat heart muscle tissue. The image shows normal mitochondria with a typical number of 15-18 cristae per mitochondria.

FIG. 11 shows a slide of over trained and inosine/orotate treated rat heart muscle tissue. The image shows enlarged mitochondria within the myocardium. Also note the larger than normal number of 20-40 cristae within the mitochondria. (22,000×)

FIG. 12 shows a slide of rat heart muscle tissue from over trained and untreated rats. Image shows mitochondria outside of the myocyte.

FIG. 13 shows a slide of heart tissue slide from over trained and untreated animals. Electron microscope slide (25,000×) of heart myocytes which have become displaced beyond the heart muscle connective tissue sheath (upper portion of slide) as a result of heart hypertrophy and dystrophy from 3 months of “overtraining.”

FIG. 14 shows a slide of heart muscle tissue from over trained and untreated rats. The image shows a damaged (possibly torn) myofibril (right center portion of the slide), a result of dystrophy from overtraining.

FIG. 15 shows a slide of heart muscle tissue from over trained and inosine/orotate treated rats. The image shows normal myocardium structures. The sarcolemma appears intact and in good condition. Also note the glycogen granules between myofibrils and normal myocardium tissue.

### DETAILED DESCRIPTION OF THE INVENTION

As used herein, the term “admixture” or “composition” means a combination of two or more components. The admixture or composition includes but are not limited to those suitable for oral, rectal, intravaginal, topical, nasal, ophthalmic, or parenteral administration to a subject. As used herein, “parenteral” includes but is not limited to subcutaneous, intravenous, intramuscular, or intrasternal injections or infusion techniques. The composition, in another embodiment, may be a beverage, such as bottled water, flavored water, soda, coffee, milk, tea, juice and the like. In the latter situation, the compound inosine or ester thereof or pharmaceutically acceptable salt thereof or combination thereof and orotic acid in acelating derivative or pharmaceutically acceptable salt thereof or combination is added to the beverage.

The term “cardioprotective” refers to any effect of the combination of inosine or pharmaceutically acceptable salt or ester thereof, and orotic acid or acylating derivative thereof or pharmaceutically acceptable salt thereof, that slows the progression of, or alleviates, the symptoms of cardiac disease. Cardioprotective also encompasses enhancing the health and performance of the heart in a subject or patient or individual that is not experiencing symptoms of heart disease. In this role, the term cardioprotective can be extended to maintaining the myocardium in sufficiently healthy condition so as to partially or completely prevent the development of cardiac disease. An additional and accurate use of the term cardioprotective would be to refer to the combination of inosine or pharmaceutically acceptable salt or ester thereof, and orotic acid or acylating derivative thereof or pharmaceutically acceptable salt thereof, as a “cardioprotective combination,” or a “cardioprotective composition.”

As used herein, “synergistically effective” means that the effect(s) observed resulting from co-administering inosine or a pharmaceutically acceptable salt or ester thereof, and orotic acid or acylating derivative thereof or pharmaceutically acceptable salt thereof, are greater than the sum of their effects when administered individually. Persons of ordinary skill in the art would readily appreciate that synergy between components may not be observed under all experimental or clinical conditions. Nevertheless, persons of ordinary skill in the art would also appreciate that synergy is not a necessary precondition for obtaining the cardioprotective benefits of the compositions and methods of use thereof, disclosed and claimed herein.

As used herein, the term “medical heart condition,” “medical heart condition of a mammalian heart” or “medical condition of a human heart” refers to a condition accompanied by diminished heart function. An example is “cardiac disease” or heart disease. The terms “cardiac disease”, “heart disease,” “cardiac insufficiency” and the like are interchangeable and more specifically describe particular symptoms, conditions and etiologies including, but not limited to heart failure, myocardial infarction, arrhythmias including angina pectoris and others, cardiomyopathy or myocardiodystrophy. In the context of the present invention, conditions “cardiac disease”, “heart disease,” “cardiac insufficiency” and the like are accompanied by an apparent functional and/or structural deterioration of the myocardium.

The present compositions contain two essential components, inosine or esters or pharmaceutically acceptable salt thereof or combination thereof and orotic acid or acylating derivative thereof or pharmaceutically acceptable salt of orotic acid or acylating derivative thereof or combination thereof.

Inosine is a nucleoside having the structure:

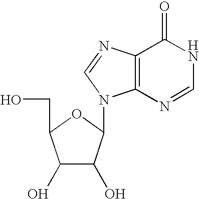

i.e., a hypoxanthine as attached to a ribose e.g. via a β-N₉ glucosidic bond. The present invention contemplates derivative of the inosine, which when ingested by the mammal, such as human, the derivative becomes hydrolyzed by the mammal to the corresponding inosine or salt thereof. For example, the inosine has three hydroxoy group, which can be esterified. Thus, as defined herein, the hydroxyl group may be esterified to form lower alkyl esters, lower alkenyl esters, lower alkynyl esters, aryl esters, aryl lower alkyl esters, cycloalkyl esters or cycloalkyl lower alkyl esters.

In another embodiment, the inosine may be in the form of phosphate (—OPO₃H) esters. It may be a monophosphate, diphosphate or triphosphate ester, which may or may not be hydrolyzed to inosine when ingested by the mammal. Inasmuch as there are tlaree hydroxyl groups, the ester functionality may be at either the 2′, 3′ or 5′ position of the sugar.

Alternatively, the present invention contemplates diesters or trimesters of inosine, wherein the ester moiety is as defined above. If a diester or triester, the ester functionality on the hydroxyl group may or may not be the same. In an embodiment, however, if a diester or triester of inosine is utilized, the ester functionalities on the hydroxyl groups are all the same.

In an embodiment, the ester functionality is on the 5′ position of the sugar ring.

The term “esters of inosine” or like expression as used herein refers to the various ester forms described hereinabove, including the phosphate esters.

Orotic acid, also known as pyrimidine carboxylic acid has the structure:

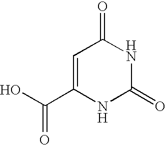

Since it is a carboxylic acid, the present invention also contemplates acylating derivatives thereof e.g. esters, anhydrides, amides, acid halides (e.g., Cl or F or Br) and the like, which are hydrolyzed in the mammal to the acids or salts. Examples of acylating derivatives include lower alkyl esters, aryl esters or aryl lower allcyl esters. Acylating derivatives of orotic acid includes compounds of the formula:

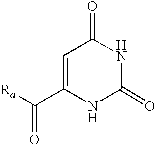

Wherein Rₐ is lower alkoxy, lower alkenyloxy, lower alkynyloxy, aryloxy, aryl lower alkoxy, cycloalkoxy or cycloalkyl lower alkoxy, wherein the cycloalkyl group contains 3-10 ring carbon atoms and up to a total of 15 carbon atoms or NR_{b}R_{c}, or —C(O)—O—C(O)— Rb, wherein Rb and Rc are independently hydrogen, lower alkyl lower alkenyl, lower alkynyl, aryl, aryl lower alkyl, cycloalky or cycloakyl lower alky, wherein the cycloalkyl group contains 3-10 ring carbon atoms, and up to 15 carbon atoms.

As used herein the term lower alkyl, when used alone or in combination, refers to a carbon chain containing 1-6 carbon atoms. It may be branched or straight-chained. Examples include methyl, ethyl, propyl, isopropyl, butyl, sec-butyl, isobutyl, tert-butyl, pentyl, neopentyl, isopentyl, hexyl and the like.

The term aryl, when used alone or in combination, as used herein, refers to an aromatic ring containing 6-14 carbon ring atoms and up to a total of 20 carbon atoms. Examples include phenyl, naphthyl anthracenyl, and the like.

Lower alkenyl, as used herein, refers to an alkenyl group containing 2-6 carbon atoms. It may have one or two or three carbon-carbon double bonds. The alkenyl group may be straight channel or branched. Examples include ethenyl, 1-propenyl, 2-propenyl, 1-butenyl, 2-butenyl, 2-methyl-1-propenyl, 1-pentenyl, 2-pentenyl, 3-pentenyl, 1-hexenyl, 2-hexenyl, 3-hexenyl and the like.

The term alkynyl, as used herein, refers to an alkynyl group containing 2-6 carbon atoms. The alkynyl group may be straight-channel or branched. Examples include ethynyl, 1-propynyl, 1-butynyl, 2-butynyl, 1-pentynyl, 2-pentynyl, and the like.

The term cycloalkyl, as used herein, refers to a cycloalkyl group containing 3-10 ring carbon atoms and up to 15 carbon atoms. The cycloalkyl group may be monocyclic, bicyclic or tricyclic and the rings may be fused. Examples include cyclopropyl, cyclobutyl, cyclopentyl, cyclohexyl, cyclohepyl, adamantyl, decalinyl and the like.

The term cycloalkyl lower alkyl, refers to a lower alkyl group, as defined herein, bonded to a cycloalkyl group, as defined herein. Examples include cyclohexylmethyl, cyclopentylethyl, and the like.

The term aryl lower alkyl refers to a lower alkyl group as defined above, bonded to an aryl group as defined above. Examples include benzyl, phenethyl, and the like.

The term lower alkoxy refers to an oxygen atom bonded to a lower alkyl group, as defined above. Examples include methoxy, ethoxy, propoxy, butoxy,sec-butoxy, t-butoxy, iso-butoxy, and the like.

Similarly, the terms, lower alkenyloxy and lower alkynyloxy refers to an oxygen atom bonded to a lower alkenyl group, as defined hereinabove, or a lower alkynyl group, as defined hereinabove respectively.

The term aryloxy refers to an oxygen atom bonded to an aryl group. Examples include phenoxy, naphthoxy, and the like.

The term aryl lower alkoxy refers to an oxygen atom bonded to a lower alkyl group as defined hereinabove, which in turn, is bonded to an aryl group, as defined hereinabove. Examples include benzyloxy, phenethoxy, and the like.

The term cycloalkoxy refers to an oxygen atom bonded to a cycloalkyl group as defined hereinabove. Examples include cyclopentyloxy, cyclohexyloxy, and the like.

The term cycloalkyl lower alkoxy refers to an oxygen atom bonded to a lower alkyl group, was defined herein, which, in turn, is bonded to a cycloalkyl group, as defined herein. Examples include cyclohexylmethoxy, cyclopentylethoxy and the like.

Each of the above groups hereinabove may be unsubstituted or may be further substituted by one or more groups or combinations of groups selected from lower alkyl or halo (e.g. F, Cl, Br, or T) hydrogen, lower alkoxy, and the like.

As defined herein, in an embodiment inosine or pharmaceutically acceptable salt is present in the combination of the present inyention. By pharmaceutically acceptable salt, it is meant those salts which are not toxic to the mammal to which they are administered. The inosine, especially the hydroxyl group or the phosphate esters thereof may be acid addition salts or pharmaceutically acceptable inorganic acids, such as hydrochloric, orthophosphoric, sulphuric, phosphoric, nitric, carbonic, boric, sulfonic, hydrobromic acids and the like as well as salts of pharmaceutically acceptable organic acids, such as acetic, propionic, butyric, tartaric, maleic, hydroxymaleic, malic, fumaric, citric, lactic, benzoic, succinic, oxalic, phenylacetic, methanesulfonic, toluenesulfonic benzenesulfonic, perchloric and the like. Alternatively, they may form salts with pharmaceutically acceptable cations, such as alkali metal or alkaline earth, or salts of other metals, or salts formed with suitable organic liquids, such as quaternary ammonium salt. Examples of pharmaceutically acceptable salts include salts of pharmaceutically acceptable cations as defined hereinabove, such as sodium, potassium, lithium, calcium, magnesium, iron, ammonium, and alkylammonium, and the like (hereinabove metal ion salts as well as ammonium salts will be referred to as inorganic salts).

Also, as defined herein, the combination may include in an embodiment, orotic acid and/or pharmaceutically acceptable. salts. Examples of pharmaceutically acceptable salts include salts of pharmaceutically acceptable cations as defined hereinabove, such as sodium, potassium, lithium, calcium, magnesium, iron, ammonium, and alkylammonium, and the like (hereinabove metal ion salts as well as ammonium salts will be referred to as inorganic salts).

In an embodiment, the pharmaceutically acceptable salts of orotic acid exclude lysine salts. In another embodiment, amino acid salts of orotic acid are excluded from the present invention.

In an embodiment, the combination comprises inosine and orotic acid or pharmaceutically acceptable salt thereof, or combination thereof, including inorganic salts, as defined herein.

The inosine, ester pharmaceutically acceptable salts thereof and the orotic acid, acylating derivatives thereof and pharmaceutically acceptable salts thereof are either commercially available or are prepared by art-recognized techniques known to the skilled artisan.

The inosine or ester or pharmaceutically acceptable salt thereof and the orotic acid or acylating derivative thereof or pharmaceutically acceptable salt thereof are present in the combination in an amount effective to treat the medical condition of the heart, as said combination being more effective than the individual compounds. In another embodiment the inosine, esters, or pharmaceutically acceptable or salt and orotic acid, or acylating derivative thereof or pharmaceutically acceptable salt thereof are present in the combination in synergistic effective amounts. As defined herein, the weight ratio refers to the amount by weight of inosine or ester thereof or pharmaceutically acceptable salt thereof divided by the weight of orotic acid, or acylating derivative thereof or pharmaceutically acceptable salt thereof. The inosine or ester thereof or pharmaceutically acceptable salt may be present in the same or lower amounts or greater amounts than the orotic acid or acylating derivative thereof or pharmaceutically acceptable salt thereof. In an embodiment, the inosine or ester thereof, or pharmaceutically acceptable salt thereof is present in a greater amount than the orotic acid or acylating derivative thereof or pharmaceutically acceptable salt thereof. In one embodiment, the weight ratio of inosine or ester or pharmaceutically acceptable salt thereof to orotic acid or acylating derivative thereof or pharmaceutically acceptable salt thereof is present in a weight ratio from about 1: 10 to about 10:1. For example, in another embodiment, the weight ratio ranges from about 1:8 to about 8:1, and in another embodiments, the ratio ranges from about 1:5 to about 5:1, while in another embodiment, the ratio ranges from about 1:4 to about 4:1, which in another embodiment, it ranges from about 2:3 to about 3:2.

As used herein, “appropriate ratio” means the weight ratios or molar ratios wherein the compounds are effective, e.g. synergistically effective ratios inosine: orotic acid or a nutraceutically or pharmaceutically acceptable salt thereof can range from approximately 1:10 to approximately 10:1, including the following approximate weight ratios of 1:8, 1:4, 1:3, 1:2, 3:4, 1:1, 4:3, 2:1 , 3:1, and 4:1, and 8:1 ofinosine to orotic acid (o a salt thereof), respectively. The physician will determine the dosage of the present therapeutic combination which will be most suitable and it will vary with the form of administration, and furthermore, it will vary with the patient under treatment, the age of the patient, and the type of malady being treated. He will generally wish to initiate treatment with small dosages substantially less than the optimum dose of the compound and increase the dosage by small increments until the optimum effect under the circumstances is reached. The combinations are useful in the same manner as comparable therapeutic agents and the dosage level is of the same order of magnitude as is generally employed with these other therapeutic agents.

The ratio of inosine or a salt thereof or an ester thereof, and orotic acid or a salt thereof or an acylating derivative thereof, as provided within a pharmaceutical or nutraceutical composition, or as dictated by adherence to a particular dosing regimen, may be altematively expressed. In this case, a calculated weight ratio is determined based on the weight of the active moieties, the inosine and the orotate, present. More specifically, the moles of inosine or salt thereof or ester thereof present is calculated based on the weight of inosine or ester or salt present and this value is multiplied by the molecular weight of inosine, which is about 268 g/mole. The number of moles of the orotic acid or acylating derivative or salt thereof multiplied by the molecular weight of the orotic acid, which is about 156 g/mole. The calculated weight ratio is the weight of the inosine moiety to orotic acid moiety present. For example, if 0.268 grams of inosine and 0.336 grams of magnesium orotate were present, the amount of the orotate moiety present is calculated by dividing the 0.336 g by the molecular weight of magnesium orotate, which is 336 g/mole and this is calculated to be 0.001 and this value is multiplied by the molecular weight of orotic acid which is 156 g/mole, and this calculation is equal to 0.156. The calculated weight ratio, in this case, is 0.336 divided by 0.156 and this is equal to about 2:1. In an embodiment, the calculated weight ratio ranges from about 1:10 to about 10:1. In another embodiment, it ranges from about 1:4 to about 4:1. Thus, the present invention contemplates calculated weight ratios of about 1:10, 1:8, 1:4, 1:3, 1:2, 3:4,4:5 1:1, 5:4, 4:3, 2:1, 3:1, 4:1, 8:1, and 10:1

This dosage regimen may be adjusted by the physician to provide the optimum preventative dosing regimen and/or therapeutic response. For example, several divided doses may be administered daily or the dose may be proportionally reduced as indicated by the exigencies of the therapeutic situation. The combination may be administered in a convenient manner, such as by oral, intravenous (where water soluble), intramuscular or subcutaneous routes. In view of how welltolerated the combination of inosine or pharmaceutically acceptable salt thereof and orotic acid, acylating derivative thereof or pharmaceutically acceptable salt thereof, when administered to subjects and patients, a composition containing the combination may be administered one or more times daily. Thus, dependent upon the subject's or patient's medical condition, a composition comprising the combination of inosine or a pharmaceutically acceptable salt thereof and orotic acid, an acylating derivative thereof or a pharmaceutically acceptable salt thereof may be administered from one to six times per day. However, this may vary dependent upon the amount of the composition taken at each administering.

Both the inosine or pharmaceutically acceptable salt or ester thereof and especially a phosphate-ester thereof, and orotic acid, acylating derivative thereof or pharmaceutically acceptable salt thereof are administered in an amount to the subject effective to treat the medical condition of the marnmalian heart. The amount to be administered of orotic acid or acylating derivative thereof or pharmaceutically acceptable salt thereof ranges from about 300 to about 8000 mg/day, while the amount of inosine or pharmaceutically acceptable salt or ester thereof, and especially a phosphate-ester thereof, ranges from about 300 to about 6000 mg/day; In another embodiment the amount of orotic acid or acylating derivative thereof or pharmaceutically acceptable salt thereof ranges from about 800 to about 5000 mg/day; while the amount of inosine or pharmaceutically acceptable salt or ester thereof, and especially a phosphate-ester thereof, and especially a phosphate ester ranges from about 800 to about 6000 mg/day. In another embodiment, especially when treating humans, the amount of orotic acid or acylating derivative thereof or pharmaceutically acceptable salt thereof ranges from about 400 to about 4000 mg/day, while that of inosine or pharmaceutically acceptable salt or ester thereof and especially a phosphate ester thereof, ranges from about 400 to about 4000 mg/day. In a further embodiment the amount of orotic acid or acylating derivative or a pharmaceutically acceptable salt thereof ranges from about 1200 to about 2000 mg/day, while that of inosine or pharmaceutically acceptable salt or ester thereof, and especially a phosphate ester thereof ranges from about 1600 to about 2500 mg/day.

As used herein, “administering” may be effected or performed using any of the methods known to one skilled in the art, which includes intralesional, icntraperitoneal, intramuscular, subcutaneous, intravenous, liposome mediated delivery, transmucosal, intestinal, topical, nasal, oral, anal, ocular or optic delivery. The compounds of the invention, e.g., inosine and orotate (orotic acid or a salt thereof), may be administered in one composition or may be administered separately (e.g., by different routes of administration, sites of injection, or dosing schedules) so as to combine in synergistically effective amounts in the subject. The dose of the composition of the invention will vary depending on the subject, the condition being treated and upon the particular route of administration used. As the usage may be for preventative measures in healthy subjects as well as for reparative purposes in subjects with existing heart conditions, dosages can range from about 0.3 grams to about 10 grams/day.

The combination may be orally administered, for example, with an inert diluent or with an assimilable edible carrier, or it may be enclosed in hard or soft shell gelatin capsules or it may be compressed into tablets, or it may be incorporated directly into the food of the diet. For oral therapeutic administration, the combination may be incorporated with excipients and used in the form of ingestible tablets, buccal tablets, troches, capsules, elixirs, suspensions, syrups, wafers, and the like. Such compositions and preparations should contain at least 1% of the combination. The percentage of the compositions and preparations may, of course, be varied and may conveniently be between about 5 to about 80% of the weight of the unit. The amount of combination in such therapeutically useful compositions is such that a suitable dosage will be obtained. Preferred compositions or preparations according to the present invention contains between about 10 mg and 6 g of the combination in synergistic amounts.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit fort is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the combination may be incorporated into sustained-release preparations and formulations. For example, sustained release dosage forms are contemplated wherein the combination is bound to an ion exchange resin which, optionally, can be coated with a diffusion barrier coating to modify the release properties of the resin.

The combination may also be administered parenterally or intraperitoneally. Dispersions can also be prepared in glycerol, liquid polyethylene glycols, and mixtures thereof, and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases, the forn must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size, in the case of dispersions, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the combination in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are the use of vacuum drying and freeze-drying techniques on the active ingredient plus any additional desired ingredients from previously sterile-filtered solutions) thereof.

As used herein, “pharmaceutically acceptable carrier” includes any and all solvents, dispersion media, coatings, antibacterial and antifungal agents, isotonic and absorption delaying agents for pharmaceutical active substances which are well known in the art. Except insofar as any conventional media or agent is incompatible with the active ingredient, its use in the therapeutic compositions is contemplated. These pharmaceutically acceptable carriers include any of the various carriers known to those skilled in the art. The following delivery systems, which employ a number of routinely used pharmaceutical carriers, are only representative of the many embodiments envisioned for administering the instant compositions. Solutions, including aqueous solutions suitable for buccal, oral, enteric, intravenous are contemplated herein. Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Oral delivery systems include tablets and capsules. These can contain excipients such as binders (e.g., hydroxypropyl-methylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid). Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “amount” means an amount sufficient to effectively treat or prevent the worsening of a medical condition of the heart, or a complication associated therewith, including enhancing the endurance or work capacity so as to prevent or reduce the occurrence or progression of the medical condition of the heart.

An embodiment of this invention provides a composition comprising an amount of inosine, a pharmaceutically acceptable salt or ester thereof and more specifically a phosphate ester thereof, and an amount of orotate (orotic acid or acylating derivative or pharmaceutically acceptable salt thereof) combined in a composition with synergistic results. In one embodiment, the amounts of inosine and orotate are in a 4:3 proportion in the composition. In another embodiment, the amounts of inosine and orotate were found to be synergistic within a weight range of from approximately 4:1 to approximately 1:4 inosine to orotate, respectively, in the composition, including, for example, specifically ratios of approximately 1:4, 1:2, 2:3, 3:4, 1:1, 4:3, 3:2, 2:1 and 4:1, inosine to orotic acid (or a salt thereof), respectively. One skilled in the art may produce the invention by combining an amount of inosine and orotic acid (which may be in the form of an orotate) in any ratio within the specified range all of which are present in synergistic amounts, as described herein, of inosine or pharmaceutically acceptable salt or ester thereof to orotic acid or acylating derivative thereof or pharmaceutically acceptable salt thereof, respectively, and delivery of the composition to the subject by any of the delivery methods discussed herein. In any of the foregoing embodiments, the composition further comprises a carrier, an excipient, an adjuvant or a combination thereof.

In one embodiment, the composition is a pharmaceutical composition and the carrier is a pharmaceutically acceptable carrier. In another embodiment, the composition is in the form of a solid dosage form, which may be a caplet, a tablet, a gelcap, a capsule or a powder.

In another embodiment, the cardioprotective combination of inosine, a pharmaceutically acceptable salt or ester thereof, and more specifically a phosphate ester thereof, and an amount of orotate (orotic acid or acylating derivative or pharmaceutically acceptable salt thereof) composition is a liquid dosage form, which may be an elixir, a syrup or linctus, or liquid mixture, a gel, an emulsion, or a suspension. The composition may be formulated as a concentrated liquid that may be diluted just prior to administering, e.g., a tincture. The compositions based on a sweet taste may substitute non-metabolized sugar substitutes for natural sugars and sweeteners. In this way, a low calorie diet may be maintained while consuming the cardioprotective composition. Just as important, diabetics may also benefit from sugar-free alternatives of beverages or snacks containing the cardioprotective composition. Various known non-metabolizable or slowly absorbed polyols may be used individually or in combination with the cardioprotective combination described herein to prepare cardioprotective beverages that are pleasing to the taste. A non-limiting list of polyols includes; e.g., mannitol, lactosucrose, sorbitol, lactitol, xylitol, maltitol, isomalt, polydextrose, and the like.

Alternatively, the composition may be provided as a component of a beverage such as vitamin waters or waters having a relatively mild amount of flavoring, or even as a soft drink, juice or juice drink, and the like. The cardioprotective properties of the combination of inosine, a pharmaceutically acceptable salt or ester thereof, and orotic acid or acylating derivative thereof or pharmaceutically acceptable salt thereof, are stable and therefore expected to have a suitable shelf life.

An embodiment includes parenteral compositions in dosage unit form for ease of administration and uniformity of dosage. Dosage unit form as used herein refers to physically discrete units suited as unitary dosages for the mammalian subjects to be treated; each unit containing a predetermined quantity of the combination calculated to produce the desired therapeutic effect in association with the required pharmaceutical carrier. The specifics for the novel dosage unit forms of the invention are dictated by and directly dependent on (a) the unique characteristics of the inosine or salt and the orotic acid or acylating derivative and the particular therapeutic effect to be achieved, and (b) the limitations inherent in the art of compounding the inosine or pharmaceutically acceptable salt thereof and the orotic acid, or acylating derivative or pharmaceutically acceptable salt thereof for the treatment of disease in living subjects having a diseased condition in which bodily health is impaired as herein disclosed in detail.

The two essential components are combined for convenient and effective administration in effective amounts with a suitable pharmaceutically acceptable carrier in dosage unit form as hereinbefore described. A unit dosage can, for example, contain the total amount of each component in amounts ranging from about 10 mg to about 6 g.

In one embodiment the inosine or pharmaceutically acceptable salt thereof and the orotic acid or acylating derivative thereof or pharmaceutically acceptable salt thereof are present in the same pharmaceutical composition.

In another embodiment, the inosine or pharmaceutically acceptable salt thereof and the orotic acid or acylating derivative or pharmaceutically acceptable salt thereof are present in a kit wherein each of inosine or pharmaceutically acceptable salt thereof and orotic acid or acylating derivative or pharmaceutically acceptable salt thereof is present in a pharmaceutical composition or nutriceutical composition. In such arrangements, the amount of inosine or ester or pharmaceutically acceptable salt thereof in one composition and the amount of orotic acid or acylating derivative or pharmaceutically acceptable salt thereof are present in the appropriate effective amounts, so that the mammal will only need to administer a defined number of a pharmaceutical composition of inosine or pharmaceutically acceptable salt thereof with a specific number of a pharmaceutical composition of orotic acid or acylating derivative or pharmaceutically acceptable salts thereof For example, the kit contains a first container containing a capsule of inosine in a pharmaceutical composition in a capsule and a second container containing a capsule of magnesium orotate in a pharmaceutical composition, wherein the weight ratio of the inosine in the first capsule to magnesium orotate in the second capsule ranges within the weight ratios defined herein, e.g. from about 10:1 to about 1:10. In such an example, the dosage administered would be a prescribed number of capsules containing inosine and a prescribed number of capsules containing magnesium orotate, e.g. one capsule of each, at any one time. When present in such a kit, the pharmaceutical composition containing inosine or pharmaceutically acceptable salt thereof salt thereof and the pharmaceutical composition containing orotic acid or acylating derivative thereof or pharmaceutically acceptable salt thereof are taken simultaneously or within about 180 minutes of each other; and in another embodiment within about 90 minutes or each other and in another embodiment within about 30 minutes of each other.

In an embodiment, the administration of the pharmaceutical or nutriceutical composition containing both components or containing only one component should be from about 1 to about 6 times during the day. Each administration of the combination, in an embodiment, is taken within a prescribed time per day, ranging from about 2 hours to about 12 hours apart and in another embodiment from about 4 to about 9 hours apart. In one regimen that may be adhered to a subject or patient administers the pharmaceutical composition from about 3-7 days to about 30 days. In an additional embodiment the treatment regimen is adhered to from about one month to about 1 year, and in yet another embodiment the treatment regimen is adhered to for more than one year. In another embodiment, especially for preventive measures, the subject will take the combination in a composition including beverage or separately, as in a kit substantially daily in the above-identified amounts for the rest of his or her life. It is noted that the precise regimen of the therapy may be open-ended according to an individual's response to the cardioprotective composition, the severity of the medical condition and associated symptoms being alleviated, or the subjeet's or patient's age, ethnicity/genetics and immediate family history and the like. As a nutriceutical, the combination of inosine or ester thereof or salt or combination thereof and the orotic acid moiety is placed in a nutriceutical carrier, such as a beverage, and mixed thoroughly. The carrier is one that is typically used in this art.

As used herein the term “patient” or “subject” refers to a warm blooded animal, preferably mammals, such as, for example, cats, dogs, horses, cows, pigs, mice, rats and primates, including humans. The preferred patient is human.

The term “treat” refers to either alleviating the cause or one or more symptom(s) of a medical heart condition, and/or preventing the medical heart condition from progressing to a more pathological or deleterious state.

This invention also provides a method of treating or preventing the onset of a cardiovascular condition in a subject by administering an effective amount of the composition of the invention to the subject thereby treating the cardiovascular condition. The cardiovascular condition to be treated may include heart failure, pulmonary hypertension, coronary heart disease, hypertensive ventricular hypertrophy, myocardial infarction, and post myocardial infarction events, all with remodeling to eccentric heart hypertrophy, arrhythmias, cardiomyopathy, myocardiodystrophy (which may include dystrophy of myocardium induced by chronic overstrain), myocarditis, pathologic remodeling of the chronically overloaded heart from many conditions including athletic overtraining and/or chronic hypertension. The administration of the composition is oral, buccal, intrabuccal, lingual, sublingual, intravenous, or subcutaneous.

The invention further encompasses embodiments directed to preventing the onset or progression of the symptoms of a medical heart condition. As used herein, the term “prevention” or prophylaxis” or similar term refers to reducing the probability of a subject from suffering from a medical heart condition, especially those who are prone by risk factors known to one of ordinary skill in the art of contracting a medical condition of the heart. Encompassed within the scope of preventing is maintaining a healthy individual's heart in a symptom-free conditions, the symptom being one or more states indicative of a cardiac disease. In another embodiment, preventing encompasses levels of prevention that are less than completely symptom-free. Thus, a treatment regimen that results in a subject or patient experiencing less than the entire repertoire of symptoms that are known in the art to characterize a given medical heart condition, is encompassed as being a preventive treatment regimen. Persons of ordinary skill in the art would appreciate that preventing, inhibiting or delaying the onset of one or more symptoms is encompassed by the treatment regimens and methods disclosed herein. The term preventing does not require a complete avoidance or absence of each symptom of a given heart condition. Therefore, it is noted that the cardioprotective combination and its method of administering is suitable as a cardio-prophylactic treatment even if the treatment does not completely or permanently delay the onset of one or more symptoms of a medical heart condition.

It is further encompassed by the present invention that a treatment regimen that increases the cardiac health or performance of a symptom-free or heart healthy individual or subject is known to be preventing or delaying the onset and severity of symptoms associated with medical heart condition. In accordance with the knowledge in the art, a heart healthy or symptom-free subject adhering to a dosing regimen that enhances the individual's physical endurance, work capacity or vital signs (e.g., EKG, VCG, X-ray, CAT scan, MRI, or various blood chemistries) is understood to be exemplifying a preventive or prophylactic administering of the pharmaceutical composition comprising the cardioprotective composition.

The administration of the composition is oral, buccal, intrabuccal, lingual, sublingual intravenous, or subcutaneous.

The invention provides a method of preventing mitochondrial exhaustion in a subject's cells comprising administering an effective amount of the composition of the invention to the subject thereby preventing mitochondrial exhaustion. The administration of the composition is oral, buccal, intrabuccal, lingual, sublingual, intravenous, or subcutaneous.

Unless indicated to the contrary, as used herein, the terms “orotic acid” and “orotate” which includes the salt and acylating derivative of orotic acid are interchangeable as they both possess the active moiety encompassed by the composition and method of invention. Similarly, specifically named salts of orotate are encompassed by these terms.

As used herein, unless indicated to the contrary, the term salt refers to pharmaceutically acceptable salt, as defined herein.

As used herein, it is to be understood, that the combination or composition may comprise one or more of the inosine or inosine esters or salts and one or more of the orotic acid or esters of salts, however, as defined herein, the combination, when administered, provides an enhanced effect, i.e., amount in treating a medical condition of the heart relative to the individual components present.

The term “combination” as used herein, refers to a combination of inosine or ester thereof of pharmaceutically acceptable salt or it may be any combination of one as more of inosine or ester thereof or pharmaceutically acceptable salt and orotic acid or acylating derivative or a pharmaceutically acceptable salt thereof or a combination of one or more of orotic acid, acylating derivative thereof or pharmaceutically acceptable salt thereof.

Unless indicated to the contrary, all amounts used herein are by weight and the ratios, as used herein are by weight ratios.

The singular denotes the plural and vice versa.

This invention will be better understood from the Experimental and Case Study Details that follow. However, one skilled in the art will readily appreciate that the specific methods and results discussed are merely illustrative of the invention.

### Experimental Results

More than 125 patients with HF have been treated with inosine/orotate combination described and claimed herein. Thus, the weight ratios described herein are non-limiting approximations that were useful as a point of beginning treatment in the most common use of the combination of inosine/orotate with human subjects and patients, the initial and finally achieved weight ratios would approximately correspond to the actual weights of the ingredient compounds, inosine and an orotate salt, as follows: 1:4 [100 mg:400 mg], 1:2 [200 mg:400 mg], 3:4 [300 mg:400 mg], 1:1 [300 mg:300 mg and 400 mg:400 mg], 4:3 [400 mg:300 mg], 2:1 [400 mg:200 mg], and 4:1 [400 mg:100 mg]. In the examples below, generally, capsules or tablets were administered with dosage ranging from two capsules to ten capsules daily taken orally.

Persons of ordinary skill in the art would readily appreciate that the weight ratios of inosine/orotate listed above are approximate. In part, this arises due to the fact that the combination has been shown to be effective for different orotate salts. These distinct salts of orotic acid will have different formula weights, therefore different amounts may be required to achieve the desired effect. The dosing regimen will depend on each subject's personal and medical history, therefore persons of ordinary skill in the art will appreciate that alterations in dosage may be implemented in less than 100 mg increments or decrements, which may provide ratios with intermittent values. Generally, alterations in the dosing would begin at week two, and revisited in either two week intervals or four week intervals, depending on the particular patient needs.

An individual with heart failure possesses a heart with significantly diminished ability to pump blood. In some cases, the heart does not fill with sufficient blood to be distributed efficiently. In other cases, the heart cannot pump blood to the rest of the body with sufficient force to provide a healthful level of oxygenation. Some individuals have both problems. As shown below, the present pharmaceutical composition was effective in overcoming these problems.

Heart failure (HF) is generally scored on a scale, from class I (demonstrable eccentric hypertrophy) to class IV (usually terminal within months). Class IV patients rarely improve and remain in class IV. Similarly, patients designated as class III tend to remain in class III and ultimately progress to Claim IV. The symptoms most often presented by patients with HF are fatigue, shortness of breath, pulmonary edema, congested liver, swollen and painful feet, and elevated levels of serum BNP (brain natriuretic peptide). BNP can be used to monitor either the improvement or progression of the disease.

In general, all of the inosine/magnesium orotate supplementation compliant patients had improved (i.e., decreased) levels of BNP (measured in pg/ml) and improvement in exercise tolerance, less shortness of breath and decreased lower extremity edema. The improvement was sufficiently marked that patients showed improvements in the overall functional class, indicating that they moved from class IV HF to class III HF and some reverted back to class II HF. This surprising result constitutes a substantial advancement in the treatment of heart failure as such results have not been observed or reported in the medical literature to date.

A notable beneficial characteristic of the compositions of the present invention and the methods of use thereof is that administering the composition to patients has not been associated with any negative indications in any of the patients under observation.

One type of cardiac disease tested with the composition and method of present invention has been cardiomyopathy, which is a devastating and unfortunate condition, the etiology of which is often undetermined.

In addition to heart failure (HF), another serious heart condition is cardiomyopathy, which is similar to heart failure in that they are both characterized by a decrease in the heart ejection fraction. The ejection fraction is a measure of left ventricular performance and is considered an index of the fraction of blood in the left ventricle that is pumped per contraction. The ejection fraction in a normal heart never actually reaches 100%, as the normal range is 63-77% for males and 55-75% for females. Morphologically, cardiomyopathy is characterized by the left ventricle wall thinning, i.e., eccentric cardiac hypertrophy. Currently, it is not known why the muscle of the heart becomes so significantly compromised.

Patients with cardiomyopathy that had their conventional medications supplemented with inosine and magnesium orotate demonstrated substantial and, in some patients, even remarkable improvement as indicated by an increase in the overall ejection fraction and restoring the contractile function of the muscle tissue. During the inventors' period of observation, many patients have been treated with the inosine and magnesium orotate composition and have observed increases in the ejection fraction ranging from approximately 20% to more than doubling, more than a 100% increase in the patients' ejection fractions. This surprising increase was obtained without significant negative indications following the inosine/magnesium orotate supplementation.

### EXAMPLES

### Example 1

Y.V. a 42 year old female with a history of coronary artery disease had been treated with a drug-eluting stent implanted into her left anterior descending (LAD) artery. Regardless of the treatment, she suffered an acute heart attack wherein the blood flow was severely compromised for approximately eight hours before being eventually restored. The majority of patients experiencing such a prolonged decrease in blood flow do not fully recover. In this case, the patient was treated with the usual medications, Plavix™, aspirin, beta-blockers and ACE iniibitors, Coumadin™, which were supplemented with the inosine/magnesium orotate supplement (400 mg/300 mg, respectively, four capsules, three times daily). Within a week of the heart attack, her recovery began to accelerate at an unusually fast pace. The patient showed no signs of heart failure or any cardiac insufficiency, and actually began exercising and biking within two weeks. Further, her energy level was remarkable for a patient who had more than 50% of her heart function compromised. Her dosage was then reduced to two capsules, twice daily. Within six weeks of the heart attack, an echocardiogram failed to show the usual signs of any previous injury. However, a nuclear stress test did show a small amount of damage in the apex of the heart.

### Example 2

An additional patient with heart failure is G.K., a 61 year old male experienced shortness of breath upon exertion. The EKG findings showed an inverted T wave suggestive of hypertrophic cardiomyopathy. Echocardiography indicated a pronounced left ventricular thickening. Without evidence of ischemic heart disease or outflow obstruction, the diagnosis of cardiomyopathy was confirmed. The patient's BNP (B-natriuretic peptide, the blood marker of HF) was elevated to 600 pg/ml. After 1 week on inosine/magnesium orotate (400 mg/300 mg, respectively) two capsules one time daily, the BNP level only slightly moderated. However, following an additional three weeks on inosine/magnesium orotate (400 mg/300 mg, respectively) with three capsules, three times daily, his BNP had dropped to 80 (normal) and he no longer experienced shortness of breath upon exertion.

### Example 3

In a case of End Stage IV CHF (heart failure), a 69 year old male patient exhibited severely decreased heart function exemplified by an ejection fraction of only approximately 10-15%. The patient received 6 capsules daily of inosine plus magnesium orotate (300 mg/300 mg, respectively, per capsule) for 6 weeks, following which his ejection fraction had improved to 15-20%.

Another cardiac condition prevalent in the population is arrhythmia. Cardiac arrhythmia is characterized by an abnormal rate of cardiac muscle contractions. The rate of contractions may be too slow, too fast, too frequent or too infrequent. The present pharmaceutical composition stabilizes the cardiac arrhythmia.

### Example 4

A patient, B.C., a 43-year-old healthy, non-smoking female with negligible intake of alcohol beverages, experienced frequent palpitations. The initial EKG showed unifocal premature ventricular beats in trigeminy (i.e., every third heartbeat was being generated from the ventricular region of the heart). An echocardiogram showed no structural or valvular abnormalities. A contrast 64 multi-slice chest CAT-scan showed completely normal coronary anatomy without any evidence of obstruction. An exercise stress test (EST) for 12 minutes showed disappearance of her abnormal ventricular premature beats at higher heart rates, but which returned soon during recovery. Twenty years ago these premature beats were treated with anti-arrhythmic drugs, which had profound side effects and in many cases resulted in an increase in mortality. The patient was started on two inosine/magnesium orotate (400 mg/300 mg, respectively) capsules two times daily to determine if there was any symptomatic relief in her premature ventricular beats. After two weeks on this regimen, the patient reported that she no longer felt the palpitations. This was confirmed upon observing that the patient's electrocardiogram had normalized. These findings were further verified with Holter monitoring (she wore an ambulatory electrocardiography device) before and after supplementation. Therefore, the inosine/magnesium orotate supplement exhibited no side effects and yet stabilized the cardiac arrhythinias.

### Example 5

A 53 year old male, D.D. with a history of elevated blood pressure and periodic episodes of arrhythmias, approximately 3-4 times weekly, began being treated daily with 50 mg Toprol for the arrhythmia and (initially) 10 mg Monopril for the blood pressure. After one month of treatment, his blood pressure had lowered from 165/105 (systolic over diastolic) to 150/95 and the arrhythmia was reduced to approximately 2-3 per week. His Monopril dosage was increased to 20 mg which resulted in his blood pressure stabilizing at 145/90, with approximately the arrhythmia frequency still at 2-3 per week. The subject's medications were then supplemented with 1 capsule/daily of a composition of inosine (300 mg) and magnesium orotate (200 mg). After two weeks his blood pressure stabilized to 135/85 but the arrhythmia decreased to 1-2 episodes per week. The inosine/magnesium orotate ratio was adjusted to 400 mg inosine and 300 mg magnesium orotate and after two weeks the arrhythmia occurrence was reduced to only once per week. Most surprising was that after the 400 mg/300 mg inosine/magnesium orotate dosage was increased to 2 capsules/day, within two weeks the blood pressure had stabilized atl 15/60 and the arrhythmias did not occur

### Example 6

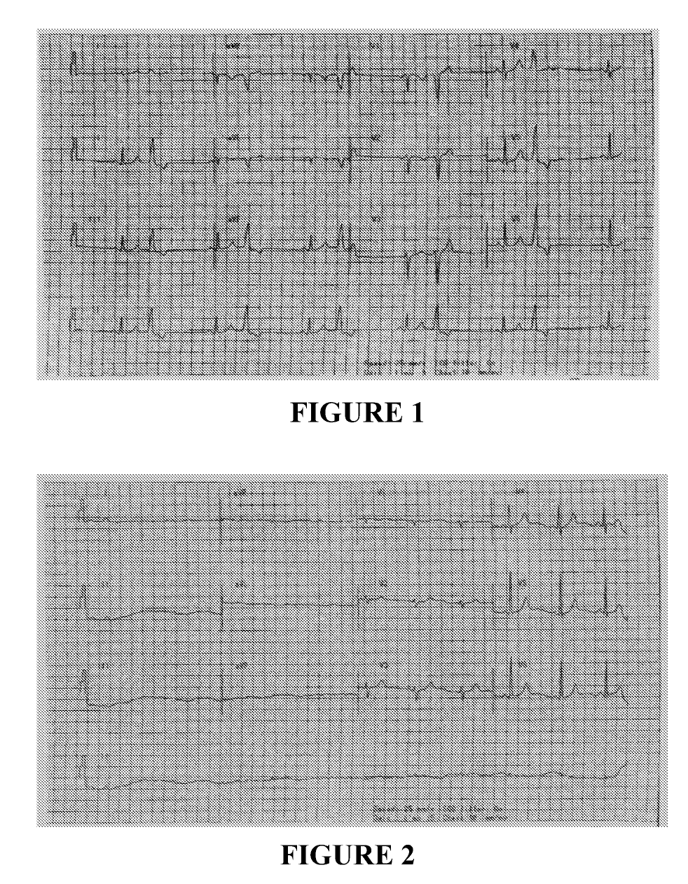

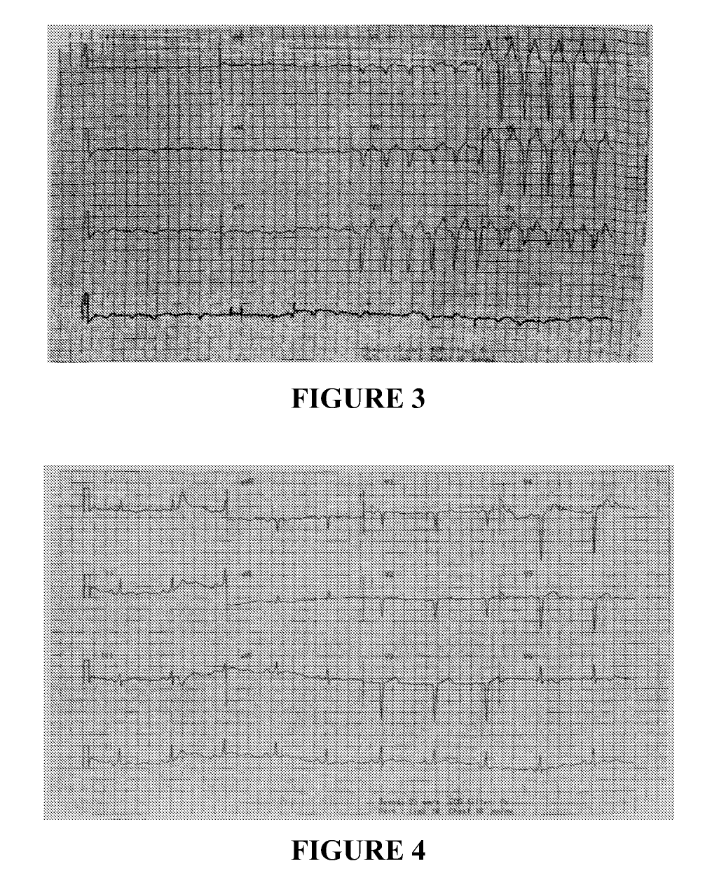

A 42 year old female patient exhibited a ventricular premature beat arrhythmia. The patient took 4 capsules daily of inosine plus magnesium orotate (400 mg/300 mg, respectively, per capsule) for 30 days, following which a stress test (Bruce protocol) indicated normal performance parameters. Further, the EKG reflected a normalization of the heartbeat (see FIGS. 1 and 2, EKG graphs.). An additional case of arrhythmia is that of a 74 year old male patient. Following treatment with 6 capsules daily for 12 days of inosine plus magnesium orotate (400 mg/300 mg per capsule, respectively) the arrhythmias were resolved (see FIGS. 3 and 4, EKG graphs).

An additional benefit of the inosine/orotate supplement is the ability to enhance the physical vitality and energy levels of those individuals having serious health problems, as shown in the following examples.

### Example 7

A 68-year-old male underwent open-heart surgery seven years prior to treatment. The subject also suffered from adult-onset diabetes, hypertension and hyperlipidemia, all of which are currently being treated with conventional medications. In the time since the bypass surgery seven years ago, he reported that he never felt as strong or energetic as he did prior to his revascularization, although he was compliant with all of his medications which normalized his blood pressure, cholesterol and diabetes. The subject always felt fatigued and became excessively tired in the early afternoons, therefore requiring frequent naps. Interestingly, despite his normal echocardiograph and myocardial perfusion scan (nuclear stress test), his quality of life had apparently deteriorated as a result of his fatigue and limited work capacity. The patient started taking inosine/magnesium orotate capsules (400 mg/300 mg, respectively, 4 capsules daily for two weeks, thereafter, 2 capsules daily). The patient reported having the energy to go on long walks, was able to fish all day, and had strength to play with his grandchildren at night. In addition, he was able to get up earlier and did not need to take naps during the day. His blood pressure was lower than it had been before the supplementation and his morning sugar levels were consistently below 100.

### Example 8

An apparently healthy 28-year-old female with no cardiac or medical history was placed on the inosine/orotate regimen after reporting that she was experiencing excessive fatigue and cramps within ten minutes of commencing to exercise or jog. A physical examination, including a routine exercise stress test (EST) was performed to evaluate her cardio-respiratory status but no abnormal condition was detected. The patient was only able to run in the stress test for 10 minutes. After being on the inosine/magnesium orotate supplement regimen (200 mg/400 mg inosine/magnesium orotate, respectively, two capsules, twice daily) for two weeks the patient reported only minimal improvement. After adjusting the ratio of inosine/magnesium orotate to 300 mg/400 mg inosine/magnesium orotate, respectively, two capsules twice daily, the patient reported being able to run for up to 30 minutes. The supplementation was not modified and following an additional two weeks, the patient reported no additional improvement. The supplement ratio was adjusted to 400 mg/300 mg inosine/magnesium orotate, two capsules, twice daily. After taking this supplement for three weeks, the patient reported a marked improvement in her stamina and was able to run for over 60 minutes without experiencing cramps or feeling overly fatigued.

Therefore, the inosine/orotate regimen increases an individual's capacity to work and exercise, whether the individual appears healthy or is known to have been suffering from serious medical conditions for a number of years.

Myocardiodystrophy represents a nonmflammatory pathology of the myocardium having more than one specific etiology. The condition is characterized by metabolic disorders in myocardium, manifested by cardiac pain that is not alleviated by nitroglycerine. It may also become more intense after alcohol consumption. It may be accompanied by shortness of breath (dyspnea), arrhythmia, and various grades of cardiac failure. A pharmaceutical composition of the present invention is successful in treating myocardiodystrophy, as the following example illustrates.

### Example 9

A 64-year-old hypertensive male was suffering from myocardiodystrophy and badly controlled diabetes and extensive triple-vessel disease with stage IV heart failure. Based on his coronary anatomy, he was not a surgical candidate. He underwent multivessel percutaneous coronary intervention (PCI/angioplasty) with multiple drug-eluting stents to help improve his health after the intervention. His exercise tolerance improved slightly. At the time of his initial evaluation, his ejection fraction (amount of blood the heart pumps on every beat) was only 20 percent (normal is 60-65 percent) and he could not walk more than 30 feet without resting. After being maximized on his heart-failure medications, his clinical course was followed. After five months, his symptoms only slightly improved. Following 7 days of 15 capsules, then 7 days of 10 capsules, then 15 days of 6 capsules of daily supplementation with inosine/magnesium orotate (400 mg/300 mg per capsule, respectively) the patient reported that he noticed a major difference and was able to walk five blocks without stopping for air. Clinically, his ejection fraction increased from 18% to 38% while his B-type natriuretic peptide (BNP) level, which was previously elevated, dropped to less than 200. Further, both his diabetes and his blood pressure became much more manageable and his overall condition has improved to stage III heart failure. This patient is one of several stage IV heart failure patients who have experienced similar results.

### Example 10

A 49 year old patient had experienced an acute myocardial infarction event, i.e., heart attack, and subsequently exhibited an ejection fraction of approximately 25%. The patient was placed on a decreasing dosage regimen of inosine plus magnesium orotate (400 mg/300 mg capsules, inosine to mag. orotate respectively) as follows: the decrease in dosage was implemented daily from Day 1 to Day 5 according to the following doses, 15 capsules, 12 capsules, 10 capsules, 8 capsules and 6 capsules. The ejection fraction improved to a level of 45% at the end of the fifth day. The dosage was then reduced to 4 capsules daily for a three month period following which the ejection fraction had further improved to 70% which is in the normal range.

Inosine is a precursor of ATP and purine molecules involved in nucleic acid (RNA and DNA) biosynthesis. Inosine has been shown individually to promote concentric hypertrophy and prevent eccentric cardiac hypertrophy in experimental aortic stenosis. At the same time, it is known that the use of orotic acid and its salts which are starting products for pyrimidine nucleic acid biosynthesis, also prevents eccentric hypertrophy and maintains the heart in a state of compensatory concentric hypertrophy. However, while the degree of concentric hypertrophy is enhanced, and/or the degree of eccentric hypertrophy is reduced, neither of these ingredients is individually able to restore full work capacity of the heart. Of interest was the possibility of preventing eccentric cardiac hypertrophy with the combined use of inosine and potassium orotate, and to determine whether the combination had cardioprotective properties or perhaps even synergistic enhancement, thereby producing the most complete energetic and positive structural changes of the myocardiurn compared with the effects of inosine or an orotate separately.

### Example 11

### Experimentally Induced Heart Failure

### A. Example 11(a)

### Short Term In Vivo Studies

There are two 7 day experiments in this portion of the study; a preliminary 7 day study with 54 rats in 9 groups (6 rats per group) to determine optimal dosage levels for the separate inosine or potassium orotate ingredients for farther experimentation by determination of maximal heart weight development and work capacity; and then a study with 30 rats in 5 groups (6 rats per group) using the optimal single ingredient dosage levels for comparison with the results of a composition of the ingredients. All compounds were administered intragastrically through a tube after being dissolved in approximately 1 ml of water.

A single control group received a sham operation, while the rest of the groups received experimental aortic stenosis (“EAS”) operations (all according to Beznak in Journal Physiol. (Lond.) 120.23P, 1953 as modified by Kogan, Bull eksper. Biol. Med., 26:112, 1961 and then the other 13 groups received various treatments and dosages of inosine or potassium orotate or a composition of the two ingredients after the optimal inosine and potassium orotate dosages were determined. All animals were tested at the beginning and end of the experiment for swimming to exhaustion times with a 7.5% body weight load attached to their tails.

### Effect of Administering Inosine and Potassium Orotate, Individually, on the Development of Heart Hypertrophy on Day Seven After Initiating Experimental Aortic Stenosis

Sach compound, i.e., inosine and potassium orotate, was administered at different doses and the level of heart hypertrophy and work capacity was observed. Each of the compounds was evaluated in four doses: 12.5 mg/kg, 25 mg/kg, 50 mg/kg and 100 mg/kg body weight. Six animals were used in each group for inosine [4 groups] and potassium orotate [4 groups] plus the one control group (sham operated, no treatment); 54 animals in total.

In this part of the experiment the control group (sham operated, no treatments) swam for 62±5.0 min. The work capacity in animals with experimental aortic stenosis, i.e., EAS, but no treatment (the “EAS Control Group”) was 15±5.0 minutes swimming time. In the EAS groups which received inosine in the above mentioned doses, the swimming times were 18±2.0; 28±3.0; 29±3.0; 27.0±3.0 minutes, respectively. In the EAS groups which received potassium orotate in the above mentioned doses, the swimming times were 18±4.0; 22±3.0; 32±3.0 and 30±3.0 minutes, respectively. All animals with experimental stenosis have significantly reduced swimming times compared to the sham operated control group. However, after just seven days of treatment, the inosine and orotate treated groups all had significantly higher swimming times than observed in the experimental stenosis group that did not receive inosine or orotate.

In this experiment on the seventh day the relative heart weight (mg per 100 gram body weight) of sham operated control group animals was 300±5.0, whereas the EAS Control Group was 400±8.9 mg/100 g body weight; in the animals with EAS which received inosine, the average heart weight was 410±9.0; 440±12, 442±12 and 440±10 mg/100 g. body weight respectively. In the EAS animals which received potassium orotate the relative weights of the hearts were 420±6.0; 440±11; 460±16 and 465±15 mg/100 g. body weight, respectively.

The data indicated that a dose of 25 mg/kg of inosine and 50 mg/kg body weight of potassium orotate was a reasonable starting point to determine the efficacy of the combined compounds in the experimental animals.

### Effect of Co-Administering Inosine and Potassium Orotate on the Ddevelopment of Heart Hypertrophy on Day Sseven After Initiating Experimental Aortic Stenosis

Thirty white rats were divided into 5 groups (6 animals in each group). Group I was a sham-operated control group, Group II animals with experimental aortic stenosis but were untreated, Groups III, IV & V animals, all with experimental aortic stenosis received by an intragastric tube each day, either inosine (25 mg/kg body weight), potassium orotate (50 mg/kg body weight) or an composition of inosine (25 mg/kg body weight) plus potassium orotate (50 mg/kg body weight), respectively.

On the seventh day after initiating experimental aortic stenosis the respective swimming times in minutes for Groups I, II, III, IV and V were 69±5.0, 17±3.0, 31±3.0, 33±4 and 46±4, and relative weights of the hearts were 305±10, 400±11, 450±12, 460±16 and 486±11 (see Table 1 below).

TABLE 1

| | | Heart | |
|---|---|---|---|
| | N^{o} of | Weight | Swimming |
| Group | Animals | mg/100 gr BW | Time (Min) |
| I | 6 | 305 ± 10 | 69 ± 5.0 |
| Sham operated | | | |
| II | 6 | 400 ± 11 | 17 ± 3.0 |
| Experimental aortic stenosis | | P¹ < 0.05 | <0.001 |
| III | 6 | 450 ± 12 | 31 ± 3.0 |
| Experimental aortic stenosis | | P¹ < 0.05 | P¹ < 0.01 |
| plus Inosine | | P² < 0.05 | P² < 0.05 |
| 25 mg/kg BW | | | |
| IV | 6 | 460 ± 16 | 33 ± 4.0 |
| Experimental aortic stenosis | | P¹ < 0.001 | P¹ < 0.001 |
| plus orotate | | P² < 0.05 | P² < 0.05 |
| 50 mg/kg BW | | | |
| V | 6 | 486 ± 11 | 46 ± 4.0 |
| Experimental aortic stenosis | | P¹ < 0.001 | P¹ < 0.001 |
| plus inosine and orotate | | P² < 0.001 | P² < 0.05 |
| 25 mg/kg and 50 mg/kg BW | | P³ < 0.05 | P³ < 0.05 |

Legend:
P¹ statistical probability of difference from Group I
P² statistical probability of difference from Group II
P³ statistical probability of difference from Group III & IV
Group I Control animals; received sham operation
Group II Control animals; received EAS operation
Group III Test animals; received EAS operation and INO treatment
Group IV Test animals; received EAS operation and PO treatment
Group V Test animals; received EAS operation and INO + PO treatment
BW Body Weight
INO Inosine
PO Potassium Orotate
NN Number of Animals
SO Sham Operated
EAS Experimental Aortic Stenosis
gr grams

The hearts of all of the groups of animals were in a condition of concentric hypertrophy, however, the inosine and potassium orotate treated animals demonstrated increased relative heart weights and increased work capacity compared to the control groups. Further, the effects of the composition of inosine and potassium orotate were significantly greater than expected compared to the treatments with either the inosine or the potassium orotate separately.

### B. Example 11(b)

### Long Term In Vivo Studies

A 12 month study using 60 white unbred male rats with initial body weights of 110-120 g, divided into four experimental groups, each group with 15 rats. Group I included intact animals (controls); Group II—rats with experimental aortic stenosis (untreated stenosed controls); Group III rats with experimental aortic stenosis given inosine in doses of 25 mg/kg/day; and Group IV rats with experimental aortic stenosis given both inosine and an orotic acid (in the instant study potassium orotate was used, however, as the orotic acid comprises 93-95% percent of any of the orotates, the results achieved are indicative of usage of any of the orotates including the potassium orotate used herein) combination in doses of 25 and 50 mg/kg/day, respectively, intragastrically, using a metal curved tube, beginning from the 45^{th} day after the creation date of the abdominal aortic stenosis, and continuing for 10.5 months. The abdominal aortic stenosis was produced by the method of Beznak, referred to hereinabove, modified by Kogan, referred to above.

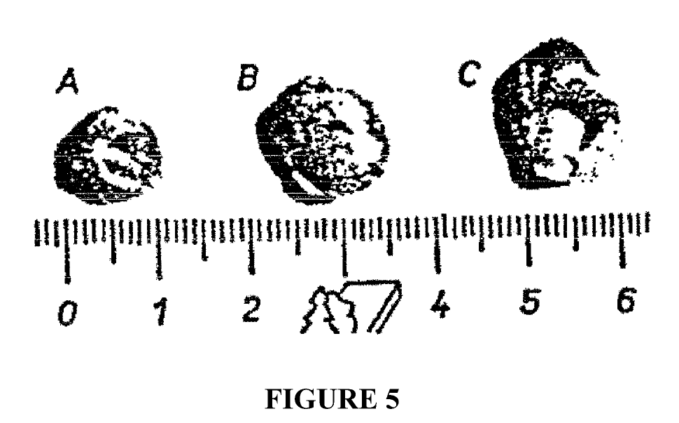

During the experimental period the animals in all four groups had regular measurements (recordings) of body weight and the 3-standard lead EKG-ms and 5-plane pericardial vector-cardiograms (VCG). At the beginning and end of the experimental period they also underwent tests for the endurance of a single, dosed physical exercise—forced swimming in a pool at 28-30° C., until maximally exhausted. Specifically, an animal had fixed to its tail a lead weight weighing approximately 7.5% percent of the animal's body weight became submerged due to the inability to continue swimming. At the end of the experimental period, the animals were sacrificed, and their hearts, thymuses, livers, adrenals and thyroids were weighed and their relative weights per 100 g body weight were calculated and recorded. Histological figures of sectioned cardiac muscle stained with hematoxilin-eosin were prepared by Van Hison's technique and examined for any attendant alterations in the normal histology of the heart. During the experiment, (untreated aortic stenosis) 8 of the 15 rats in Group II died, while in Group III (treated stenosis w/inosine) and Group IV (treated stenosis w/inosine and potassium orotate)—only 3 rats in each group died. It is noted, that by the end of the experimental period, animals in Group II showed marked slowing of the growth rate (41.5% percent lower weight gain than that of the intact control rats in Group I). The observed time period wherein the rats could swim were as follows: Groups I animals 45±2.0 minutes; Group II 6.5±2.1 minutes; Group III 16±1.2 minutes; and Group IV 32±4 minutes, respectively. Thus, the group receiving the combination of inosine and potassium orotate performed far better than Group II (untreated stenosis) and had doubled the work capacity of Group III (treated with inosine only). The relative weight of the heart in animals in Groups II, III and IV was higher than in Group I (control group) by 67%, 36% and 56%, respectively. (See Tables 2 A-B). The hearts of Group II animals were in the state of eccentric hypertrophy which explains the higher weight (see FIG. 5). The left ventricular wall in the animals of Group II was 8% thinner than in rats of Group I as a result of the eccentric condition. In rats of Groups III and IV the left ventricular wall was 30% and 46% thicker, respectively, than the rats in group I and they were all in the state of concentric heart hypertrophy.

TABLE 2-A

| | No. of | | | | | | | | | | |
|---|---|---|---|---|---|---|---|---|---|---|---|
| | animals | | | | Working capacity | | Relative weights of | | | | Thickness |
| | at 1½ | | Body weight (g) | | (min) | | organs (mg/100 g) 12 | | | | of left |
| Group | 12 | Mortality | 1½ | 12 | 1½ | 12 | months after surgery | | | | ventricular |
| of Animals | months | % | months | | months | | Heart | Thymus | Liver | Adrenals | wall (mm) |
| I Intact | | | | | | | | | | | |
| No aortic | 15 | 0 | 200 ± 15¹ | | 52 ± 3.0 | | | | | | |
| stenosis | | | | | | | | | | | |
| | 15 | | | 472 ± 7¹ | | 45 ± 2.0 | 284 ± 4.0 | 122 ± 2.3 | 2600 ± 663 | 9 ± 0.5 | 3.0 ± 0.1 |
| II Experimental | 15 | 47 | 195 ± 4.0 | | 48 ± 5.2 | | | | | | |
| aortic stenosis | 8 | | | 390 ± 15 | | 6.5 ± 2.1 | 477 ± 11 | 78 ± 7.8 | 2993 ± 219 | 7 ± 0.4 | 2.76 ± 0.20 |
| | | | P¹ > 0.7 | P¹ < 0.001 | P¹ > 0.5 | P¹ < 0.001 | P¹ < 0.001 | P¹ < 0.001 | P¹ > 0.5 | P¹ < 0.001 | P¹ > 0.4 |
| | | | | | | | 695 ± 10² | 77 ± 1.5² | | | |
| III | 15 | 20 | 198 ± 6.0 | | 43 ± 4.6 | | | | | | |
| Experimental | | | | | | | | | | | |
| aortic stenosis | 12 | | | 443 ± 12 | | 16 ± 1.3 | 387 ± 9.5 | 116 ± 7.5 | 3475 ± 148 | 11 ± 0.7 | 3.9 ± 0.14 |
| +inosine 25 | | | | | | | | | | | P¹ < 0.001 |
| mg/kg | | | | | | | | | | | |
| | | | P¹ > 0.8 | P¹ < 0.06 | P¹ < 0.2 | P¹ < 0.01 | P¹ < 0.001 | P¹ > 0.6 | P¹ > 0.7 | P¹ < 0.05 | P² < 0.001 |
| | | | P² > 0.7 | P² < 0.01 | P² < 0.05 | P² < 0.001 | P² < 0.001 | P² < 0.001 | P² > 0.05 | P² < 0.001 | |
| Group IV | 15 | 20 | 200 ± 5.0 | | 47 ± 17 | | | | | | |
| Experimental | | | | | | | | | | | |
| aortic stenosis | 12 | | P¹ - NS | 460 ± 12 | P¹ - NS | 32 ± 4.0 | 440 ± 14 | 120 ± 5.6 | 3695 ± 104 | 10 ± 0.3 | 4.4 ± 0.12 |
| +inosine 25 | | | P² - NS | P¹ < 0.001 | | P¹ < 0.05 | P¹ < 0.05 | P¹ - NS | P¹ - NS | P¹ - NS | P¹ < 0.05 |
| mg/kg BW plus | | | | | | | | | | | |
| potassium | | | P³ - NS | P² < 0.01 | P² - NS | P² < 0.05 | P² > 0.05 | P² < 0.05 | P² < 0.05 | P² < 0.05 | P² > 0.05 |
| orotate | | | | | | | | | | | |
| 50 mg/kg BW | | | | | | | | | | | |
| | | | | P³ < 0.01 | P³ - NS | P³ < 0.05 | P³ < 0.05 | P³ < 0.05 | P³ < 0.05 | P³ - NS | P³ < 0.05 |

LEGEND

| | |
|---|---|
| P¹ | statistical probability of difference from Group I |
| P² | statistical probability of difference from Group II |
| P³ | statistical probability of difference from Group III |
| BW | Body weight |
| g | grams |
| min | minutes |
| NS | not significant |

The prolonged administration of inosine and the inosine−potassium orotate combination prevented development of cardiac eccentric hypertrophy in the rats of Groups III and IV, respectively, and promoted a 30% and 40% percent increase in the thickness of the left ventricular wall of Groups III and IV, respectively, when compared to Group I (control). (See Table 2-A). The relative heart weight in Group II animals exceeded the critical weight of rats with the same body weight by approximately 31% (established in rats subjected to long-term training) (see Table 7-A). (The critical heart weight is the maximal heart weight at which the heart is still in a state of compensatory concentric heart hypertrophy (see FIG. 5)). In comparison, the critical weights in rats in Groups III and IV were enhanced by 16% and 26% respectively. FIG. 5 provides a macroscopic view of the normal heart of a rat (A), while FIG. 5C exemplifies a heart having eccentric hypertophy wherein aortic stenosis was experimentally induced, and FIG. 5B exemplifies a heart having concentric hypertrophy wherein aortic stenosis was induced and the rat received an inosine supplement.

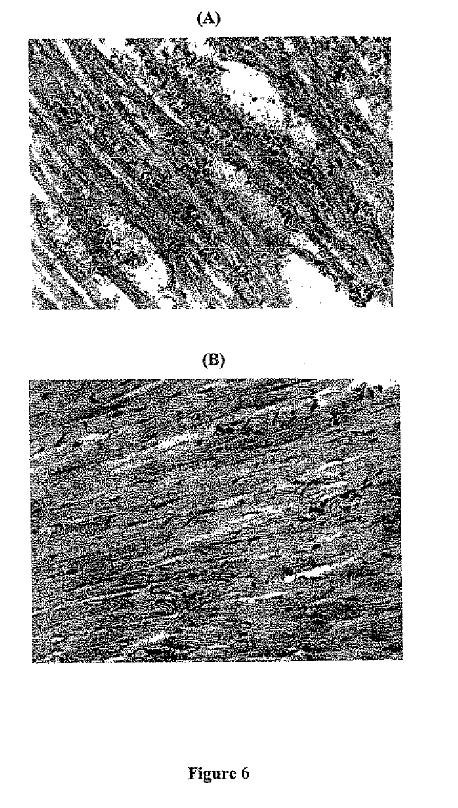

Histological examination of the myocardium from Group II rats revealed disordered circulation with areas of diffuse hemorrhage, and formation of oval blood-filled cavities in the intramuscular spaces. Apart from this, irregular hypertrophy of cardiac muscular fibers was seen. A substantial proportion of the fibers were in an atrophic state, and their cross-striated pattern was not clear. The cytoplasm in individual cells was unevenly stained with eosin and vacuolated. The myocardial nuclei were predominantly small in size, hyperchromic, and displaced to the cell periphery. The cardiac interstitium showed accumulation of lymphoid elements and areas of myocardiosclerosis. The walls of larger vessels were thickened, loosened, and their muscle coats were hypertrophic. (See FIG. 6-A).

In the hearts from Groups III and IV rats, i.e., rats treated with inosine or inosine lonotate, no circulatory or vascular abnormalities were observed. Muscle fibers were hypertrophic and had a distinct cross-striated pattern, and they were evenly stained with eosin. Hypertrophic, transparent or moderately hyperchromic myocardial nuclei were mostly located in the central portion of fibers. Myocardiosclerotic areas were only occasionally observed. (See FIG. 6-B). However, as shown by the data in Table 2-A, the effect is significantly greater in Group IV, for example, the working capacity was greater when the rat was treated with low inosine/orotate than when treated with inosine alone.

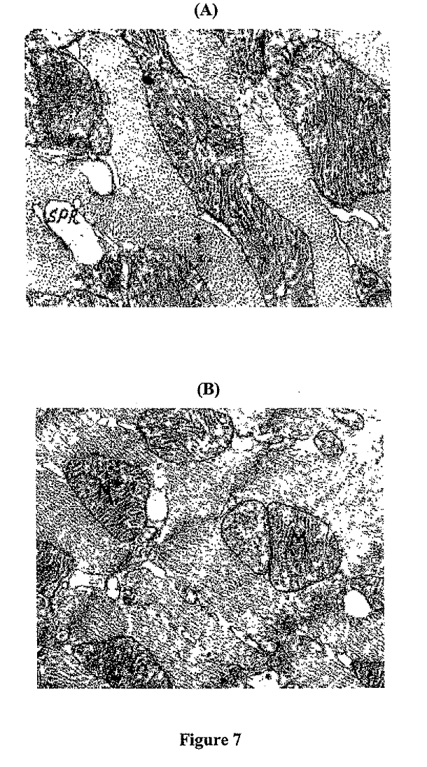

Thus, prolonged cardiac hyperfunction due to the experimental aortic stenosis in rats resulted in the development of eccentric heart hypertrophy with its characteristic morphologic alterations in the myocardium. In contrast, the use of inosine or an inosine−potassium orotate combination during the 10.5 month experimental period prevented these alterations, and in fact significantly increased the concentric heart hypertrophy (see FIGS. 6-7). For example, the ventricular wall thickness and relative heart weight increased in parallel with significant increased work capacity (forced swimming time) when compared to the other groups. As shown in FIG. 7, in the untreated rat, the mitochondria was distended, however, when treated with inosine or the combination of inosine/orotate, the mitochondria of those rats were no longer distended, but were normal size. Futher, as indicated by the data, the working capacity of the heat in those rats receiving the combination of inosine/orotate was significantly greater than that of the heart in those rats receiving inosine alone.

The electrophysiological studies of Group II revealed a considerable overload of the heart ventricles. This manifested itself with marked tachycardia, polytopic and often successive extrasystoles, distinct lengthening of the PQ- and QRS segments, higher systolic index, low T waves amplitude and an upward shift of ST segments in the EKGs. The vector cardiograms, i.e., VCGs, indicated development of pathologic hypertrophy of the heart, judging from the open QRS- and T-loops, a decreased T loop amplitude, and discordance of QRS- and T-angles. In contrast, only a few animals in Group III, and none in Group IV demonstrated these abnormalities. Therefore the EKG- and VCG-evidence taken together suggests substantially less pathological alterations in myocardial metabolism in the Groups III and IV (Table 2-B).

TABLE 2-B

| | No. of | — | Heart Rate | | EKG Findings (1 mV = 10 mm) | | | | | |
|---|---|---|---|---|---|---|---|---|---|---|
| | animals | Mor- | (beats/min) | | Stroke Index (%) | | QRS (sec) | | R Wave Voltage (mV) | |
| Group of | at 1½ | tality | 1½ | 12 | 1½ | 12 | 1½ | 12 | 1½ | 12 |
| Animals | 12 months | % | months | | months | | months | | months | |
| Group I Intact | 15 | 0 | 400 ± 20 | | 33 ± 3.2 | | 0.015 ± 0.001 | | 0.56 ± 0.05 | |
| No Aortic | 15 | | | 428 ± 17 | | 34 ± 2.1 | | 0.021 ± 0.001 | | 0.51 ± 0.02 |
| Stenosis | | | | | | | | | | |
| Group II | 15 | 47% | 391 ± 17 | | 50 ± 2.3 | | 0.014 ± 0.002 | | 0.30 ± 0.06 | |
| Experimental | | | | | | | | | | |
| Aortic Stenosis | 8 | | | 473 ± 22 | | 55 ± 1.8 | | 0.031 ± 0.002 | | 0.38 ± 0.03 |
| | | | P¹ > 0.7 | P¹ > 0.1 | P¹ < 0.00 [symbol] | P¹ < 0.00 [symbol] | P¹ > 0.6 | P¹ > 0.5 | P¹ < 0.01 | P¹ < 0.01 |
| Group III | 15 | 20% | 410 ± 11 | | 47 ± 10 | | 0.021 ± 0.004 | | 0.38 ± 0.05 | |
| Experimental | | | | | | | | | | |
| Aortic Stenosis | 12 | | | 420 ± 16 | | 34 ± 1.2 | | 0.022 ± 0.001 | | 0.9 ± 0.09 |
| + Inosine | | | | | | | | | | |
| 25 mg/kg BW | | | | | | | | | | |
| | | | P¹ > 0.7 | P¹ > 0.6 | P¹ < 0.00 [symbol] | — | P¹ > 0.2 | P¹ > 0.5 | P¹ < 0.02 | P¹ < 0.001 |
| | | | P² > 0.03 | P² > 0.05 | P² < 0.03 | P² < 0.00 [symbol] | P² > 0.2 | P² > 0.05 | P² > 0.04 | P² < 0.001 |
| Group IV | 15 | 20% | 412 ± 11 | | 48 ± 4 | | 0.023 ± 0.002 | | 0.31 ± 0.05 | |
| Experimental | | | | | | | | | | |
| Aortic Stenosis | 12 | | | 421 ± 14 | | 36 ± 1.4 | | 0.020 ± 0.001 | | 0.93 ± 0.09 |
| + Inosine | | | | | | | | | | |
| 25 mg/kg BW | | | | | | | | | | |
| potassium | | | P¹ - NS | P¹ - NS | P¹ < 0.00 [symbol] | P¹ - NS | P¹- NS | P¹- NS | P¹ < 0.01 | P¹ < 0.001 |
| orotate | | | | | | | | | | |
| 50 mg/kg BW | | | | | | | | | | |
| | | | P² - NS | P² < 0.05 | P² - NS | P² < 0.01 | P² - NS | P² < 0.05 | P² - NS | P² < 0.001 |
| | | | P³ - NS | P³ - NS | P³ - NS | P³ - NS | P³ - NS | P³ - NS | P³ - NS | P³ - NS |

LEGEND:

| | |
|---|---|
| P¹ | statistical probability of difference from Group I |
| P² | statistical probability of difference from Group II |
| P³ | statistical probability of difference from Group III |
| BW | Body weight |
| g | grams |
| min | minutes |

T Wave Voltage, Index (%), QRS (sec), R Wave Voltage (mV0, (mV0, QRS, T and QRS-T are Standard EKG measurement designations

| | |
|---|---|
| VGC | Vectographic cardiogram |

[symbol]

indicates data missing or illegible when filed

Comparisons of anatomical, morphologic and VCG evidence failed to establish correlations between the patterns of change in the depolarization and repolarization phases in VCG loops, and the absolute weights and wall thicknesses of the heart specimens that were examined. The subjects in Groups III and IV presented almost no signs of the pathologic hypertophy, whereas 100 percent of the animals in Group II did so. Swimming times for rats in Groups I, III and IV, with normal cardiac metabolism (as inferred from the VCG-recordings), were significantly longer than those of animals with compromised heart metabolism (Group II) (Table 2-C).

TABLE 2-C

| | No. of | | | | VGC Findings (1 mV = 20 mm) after 12 | | | | QRS-T |
|---|---|---|---|---|---|---|---|---|---|
| | animals | | T Wave Voltage | | months 12 months after surgery | | | | angle |
| | at 1½ | | (mV) | | QRS | | | T | 120° |
| Group of | 12 | | 1½ | 12 | Not closed | Amplitude | Area | Amplitude | to |
| Animals | months | Mortality % | months | | (%) | (mV) | (cm²) | (mV) | 180° |
| Group I Intact | 15 | 0 | 0.16 ± 0.02 | | 20 | | | | |
| No Aortic | 15 | | | 0.20 ± 0.02 | | 1.6 ± 0.14 | 4.6 ± 0.6 | 0.5 ± 0.02 | 1 |
| Stenosis | | | | | | | | | |
| Group II | 15 | 47% | 0.23 ± 0.02 | | 87.5 | | | | |
| Experimental | 8 | | | 0.16 ± 0.04 | | 2.8 ± 0.1 | 12.6 ± 1.2 | 0.3 ± 0.01 | 7 |
| Aortic Stenosis | | | P¹ < 0.01 | P¹ > 0.6 | | P¹ < 0.001 | P¹ < 0.001 | P¹ < 0.001 | |
| Group III | 15 | 20% | 0.21 ± 0.01 | | 58 | | | | |
| Experimental | 12 | | | 0.29 ± 0.05 | | 3.2 ± 0.12 | 8.3 ± 1.1 | 0.6 ± 0.01 | 5 |
| Aortic Stenosis + | | | P¹ < 0.05 | P¹ > 0.2 | | P¹ < 0.001 | P¹ < 0.001 | P¹ < 0.001 | |
| Inosine 25 mg/kg | | | P² > 0.2 | P² < 0.05 | | P² < 0.02 | P² < 0.02 | P² < 0.001 | |
| BW | | | | | | | | | |
| Group IV | 15 | 20% | 0.22 ± 0.02 | | 30 | | | | |
| Experimental | 12 | | | 0.33 ± 0.05 | | 3.6 ± 0.10 | 8.9 ± 1.2 | 0.7 ± 0.01 | 3 |
| Aortic Stenosis + | | | P¹ < 0.05 | P¹ < 0.05 | | P¹ < 0.05 | P¹ < 0.05 | P¹ < 0.05 | |
| Inosine 25 mg/kg | | | P² < 0.05 | P² < 0.05 | | P² < 0.05 | P² | P² < 0.05 | |
| BW | | | P³ < 0.05 | P³ < 0.05 | | P³ < 0.05 | P³ | P³ > 0.05 | |
| potassium | | | | | | | | | |
| orotate 50 mg/kg | | | | | | | | | |
| BW | | | | | | | | | |

LEGEND:

| | |
|---|---|
| P¹ | statistical probability of difference from Group I |
| P² | statistical probability of difference from Group II |
| P³ | statistical probability of difference from Group III |
| BW | Body weight |
| g | grams |
| min | minutes |

T Wave Voltage, Index (%), QRS (sec), R Wave Voltage (mV0, (mV0, QRS, T and QRS-T are Standard EKG measurement
designations

| | |
|---|---|
| VGC | Vectographic cardiogram |

Taken together, the experimental evidence indicates that prolonged administration of the inosine−potassium orotate composition in rats experiencing experimentally induced aortic stenosis effectively promoted compensatory concentric hypertrophy of the heart and beneficial increases in the weight of the heart within the critical heart weight limits and thereby prevented the development of eccentric cardiac hypertrophy.

### Example 12

### Myocardial Infarction

A myocardial infarction is the condition of development of a necrotic area in the heart as a result of the occlusion of coronary vessels. This study establishes that a composition of inosine and an orotic acid promotes beneficial post-infarction processes, such as forming a post-infarction scar with a collateral correcting effect on the state of the peri-infarction zone. The composition of the present invention also promotes the intensified lysis of the necrotized tissue and replacement of the necrosis zones with connective tissue and collagen. At the same time, resorption of necrotic masses is considerably accelerated with simultaneous rapid filling of the infarcted region with cellular elements of the connective tissue and accelerated formation, in this region, of a dense-elastic scar. Thus, the inosine/orotate composition accelerates formation of the post-infarction scar, thus improving conditions for heart muscle functioning, and increased myocardial oxygenation in the perinecrotic zone and (during the initial stage of the disease) in the extra-infarctional areas of myocardium.

The composition according to the present invention has been experimentally tested on 90 chinchilla rabbits experiencing experimentally-induced myocardial infarction produced by the ligation of the front descending branch of the coronary artery. The effect of a composition of inosine and orotic acid composition 100 mg/kg/body weight on the progression of the experimental myocardial infarction has been studied in comparison with the effect produced by inosine or orotic acid used separately.

Histological, light microscopic methods of investigation have been employed to study the zone of necrosis, perinecrotic zone and myocardium regions spaced from the time of infarction by 7 and 14 days after the infarction event. Dynamic electrocardiographic observation over the animals was carried out during the first day and during the following 7 and 14 days. The study of the total cross-sectional patterns of a rabbit's heart has made it possible to precisely define these zones.

The first Group of 10 animals was sham-operated without treatment (Group I, positive control-Intact, no MI). All 80 remaining animals were operated on to create myocardial infarction (MI) and were then randomly divided into Groups II, III, IV and V with 20 animals in each group. Each of these groups was further divided into two groups of 10 animals each, for 7 and 14 day testing, respectively.

Group II animals were without treatment (negative control-MI Control). Group III animals received orotic acid in an oral dose of 100 mg/kg/body weight every day during 7 and 14 days. Group IV received inosine in an oral dose of 100 mg/kg/body weight every day during 7 and 14 days. Group V received the combination composition—inosine plus orotic acid in an oral dose of 100 mg/kg/body weight of each ingredient (200 mg/kg/body weight total) every day during 7 and 14 days.

No changes in the EKG could be detected in the Group I control (non-operated on) rabbits observed for 14 days. The second day after the operation, the Group II, III, IV, and V rabbits showed changes characteristic of acute myocardial infarction: displacement of S-T segment downwards from the isoelectric line, decrease and deformity or appearance of a discordant T wave, the pathological Q wave and displacement of the S-T interval upwards from the isoelectric line. Sometimes the QRS complex was changed dramatically and looked as one R wave pointed downwards. The reparative processes in the myocardium were accelerated under the effect of the orotic acid (Group III), the inosine (Group IV) and the inosine and orotic acid composition (Group V) and could also be evaluated from electrocardiographic data (see Table 3).

On the second day after the operation, the heartbeat rate increased by 8-12%, the voltage of the EKG waves diminished, while the systolic index (ratio of QRS complex to ST complex in standard EKG terms] rose by 15-16%.

The heartbeat rate returned to the norm in the Group II control animals by the fourteenth experimental day. At that time the Group II control animals showed a progressive deepening of the Q wave, starting from the second day and continuing up to the fourteenth day after the operation, i.e. the normal time course of changes in the EKG readings observed after experiencing an extensive infarction. No restoration of the systolic index was seen even by the fourteenth day.

TABLE 3

| | | | | | | | | Collagen per | |
|---|---|---|---|---|---|---|---|---|---|
| | | | EKG DATA | | | | Thickness of | dry tissue weight | |
| Group | | Number of | Q | R | QRS | NN of Animals | Left Ventricular | (mg/gram) | |
| of Animals | | Animals | mm | mm | sec | With Necrosis | Wall (mm) | Soluble | Non-Soluble |
| I | Day 7 | 10 | 1.7 ± 0.11 | 3.9 ± 0.2 | 0.08 ± 0.005 | — | NOT EVALUATED | | |
| (Intact, no MI) | Day 14 | same 10 | 1.8 ± 60.12 | 3.8 ± 0.1 | 0.07 ± 0.005 | 0 | 5.25 ± 0.028 | 32.1 ± 3.5 | 24.0 ± 3.0 |
| II | Day 7 | 10 | 2.4 ± 0.01 | 2.2 ± 0.2 | 0.18 ± 0.003 | 10 | 4.15 ± 0.48 | 21.0 ± 1.2 | 7.01 ± 2.0 |
| Control (MI) | Day 14 | 10 | 2.8 ± 0.7 | 2.4 ± 0.16 | 0.12 ± 0.006 | 3 | 4.63 ± 0.66 | 32.4 ± 2.4 | 18.1 ± 2.0 |
| III | Day 7 | 10 | 1.0 ± 0.02 | 4.2 ± 0.030 | 0.07 ± 0.003 | 5 | 5.0 ± 0.61 | 31.1 ± 1.9 | 15.2 ± 1.6 |
| MI + OrA | Day 14 | 10 | 0.6 ± 0.05 | 4.4 ± 0.27 | 0.06 ± 0.016 | 0 | 5.4 ± 0.23 | 41.7 ± 1.9 | 27.2 ± 1.5 |
| 100 mg/kg BW | | | | | | | | | |
| IV | Day 7 | 10 | 1.1 ± 0.03 | 4.1 ± 0.30 | 0.7 ± 0.003 | 5 | 5.2 ± 0.72 | 30.0 ± 2.0 | 16.1 ± 2.1 |
| MI + INO | Day 14 | 10 | 0.8 ± 0.02 | 4.3 ± 0.28 | 0.068 ± 0.003 | 0 | 5.6 ± 0.21 | 42.0 ± 3.0 | 28.2 ± 1.5 |
| 100 mg/kg BW | | | | | | | | | |
| V | Day 7 | 10 | 0.6 ± 0.05 | 4.6 ± 0.22 | 0.065 ± 0.002 | 1 | 6.2 ± 0.44 | 37.1 ± 2.5 | 21 ± 3.2 |
| MI + INO + OrA | Day 14 | 10 | 0.4 ± 0.016 | 4.6 ± 0.3 | 0.066 ± 0.002 | 0 | 6.0 ± 0.35 | 44 ± 3.0 | 28.0 ± 3.0 |
| 100 mg/kg BW each | | | | | | | | | |

LEGEND:

| | |
|---|---|
| MI | Myocardial Infarction |
| BW | Body Weight |
| OrA | Orotic Acid |
| INO | Inosine |
| NN | number of animals |
| EKG | electrocardiogram |
| Q, R, QRS | Standard electrocardiogram measurement designations |
| Group I | Intact animals, no MI operation, no treatments |
| Group II | Received MI operation, no treatments |
| Group III | Received MI operation, and Orotic Acid Treatment |
| Group IV | Received MI operation and Inosine Treatment |
| Group V | Received MI operation, and Inosine and Orotic Acid |
| | Treatment |

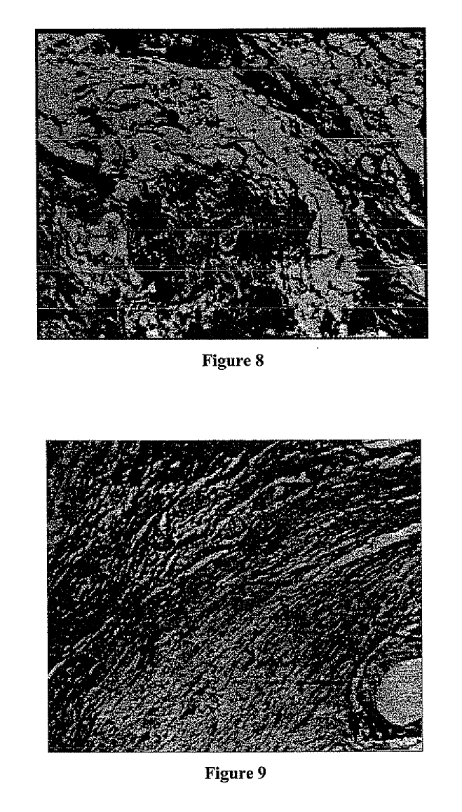

In contrast to the Group II (control animals), those given orotic acid or inosine, and the combined composition of both demonstrated positive changes in all the EKO readings examined. By the seventh experimental day all the animals treated with orotic acid or inosine, or the combination composition showed nearly a complete restoration of the voltage of EKG waves and of the systolic index. No deepening of Q wave was detected on days 7 and 14 after the operation, as compared with that observed on the second day. Especially suggestive was the rapid time course of changes in the S-T segment and the T wave, indicating effective cicatrization (i.e., process of scar formation) in the zone of necrosis (Table 3). This interpretation was supported by post-mortem morphological examination. Thus, the rapid time course of the EKG readings in the groups receiving either inosine or orotate or the combined composition is indicative of an activation of the reparative processes in the zone of the infarction and to the segregation of the necrotic area in the animals exposed to experimental therapy. See FIGS. 8 and 9.

The morphological appearance of the zone of infarction in Group II (untreated MI animals) was also different from the treated experimental animals in Groups III, IV and V. The control animals developed the infarction which involved the entire anterior wall of the left ventricle, the anterior third of the interventricular septum, part of the front papillary muscle, and partly the anterior wall of the right ventricle. Sometimes the infarctions penetrated into all layers of the myocardium. Group II control animals with extensive myocardial infaretions showed disseminated necroses on the seventh experimental day. On the fourteenth experimental day the necroses were detected in 50% of the animals.

In contrast, the administration of orotic acid or inosine individually, resulted in a 50% reduction in necroses. Surprisingly, the animals receiving the inosine plus orotic acid combination demonstrated necrosis elimination in 90% of the animals by the seventh day. This represents a substantial improvement of 80% over the individual ingredient treated animals. It is further surprising to find that by the fourteenth day, necrosis elimination was observed in all of the animals. It is also noteworthy that in the animals treated with the inosine/orotate combination the areas of the myocardium where necrosis occurred were smaller, approximately 25% of the size observed in the controls [Group II]. The experimental rabbits had a more pronounced (as compared with the controls) increase in the number of poorly-differentiated young intensely basophilic fibroblasts and less marked infiltration in the area of the cicatrix being formed and in the adjacent areas. These rabbits developed no hemorrhages and showed vascular neofornation. See FIGS. 1 and 2.

By the fourteenth day, the treated rabbits (Groups III, IV and V) exhibited further differentiation of the cellular and fibrous elements of the connective tissue in the infarction zone. The collagen fibers looked like thick fascicles oriented along the continuation of the stumps of the muscular fibers from the defect edges towards the central part. By fusing with the fibers lying at the opposite side they began to cover the defect. The zone of infarction and the remainder of the myocardium demonstrated a higher content of mature cellular-fibrous elements, as compared with the controls. By that time all the animals treated with orotic acid, inosine or the combination composition virtually had no hemorrhages or infiltration in the infarction zone.

Thus, the animals given orotic acid or inosine, and to an even greater degree the combination composition, demonstrated a higher activity of cicatrization and vascular neoformation in the zone of infarction. In addition, the treated animals provide substantially less infiltration and restricted hemorrhages in comparison to the control animals.

In all of the experiments the activity of the combination composition of inosine and orotic acid was significantly more effective than activity of inosine or orotic acid used separately (Table 3). The anatomical evaluation of hearts of the animals showed that the ventricular thickness in Group V was significantly thicker than in Groups I, II, III and IV. This evidence proves the higher level of compensatory hypertrophy in this group of animals compared with Groups II, III and V (Table 3).

Resorption of necrotic tissue demonstrated the higher rate in animal Groups III, IV and V compared with Group II. However, the combination composition of inosine and orotic acid clearly provided the most superior result as determined by EKG data and histological observations of activity of the resorption of necrotic tissue. This result is further confirmed by evaluation of the concentration of soluble and non-soluble collagen in areas of infarction among the control and experimental groups as shown in the Table 3. By day 7 it was evident that insoluble collagen had accumulated in significantly higher amounts at the infarction sites of Group V in comparison to Groups IV, III and I. The concentration of non-soluble collagen is a measure of the durability of the scar in the area of the necrosis.

The physiological and histological data provides substantial corroborating evidence of the benefits of co-administering combinations of inosine and orotic acid to alleviate, reverse various causes and/or symptoms of cardiac insufficiency. Pathological heart conditions such as myocardial infarction, arrhythmia, heart failure, and myocardiodystrophy can be effectively treated. Further, the combination of inosine and orotic acid and its method of use has been shown to be virtually completely free of undesirable side effects of the treatment for long periods of time and thus renders it suitable for long-term prophylactic use.

### Example 13

### Myocardiodystrophy

Myocardiodystrophy represents a noninflammatory pathology of the myocardium characterized by metabolic and energetic irregularities or disorders in the myocardium. In human patients, it may be manifested by morning cardiac pain that is not alleviated with nitroglycerine, and it may become more intense after alcohol consumption. Other indications are dyspnea, arrhythmia and diverse grades of cardiac failure.

In recent years a significant amount of attention has been focused on an EKG syndrome referred to as chronic cardiac overstrain (CCOS), which can be found in individuals that participate in high performance sports. It is known that in the course of training some athletes will develop EKG alterations that may are characterized by modifications of the end part of the ventricular complex of an athlete's EKG. This observation suggests that ath letes are at risk of developing myocardial dystrophy as a result of prolonged physical overexertion.

Fifty white rats having an average body mass of between 180-200 g at the start of the experiment were used for the study over a three month period. Depending on the experimental group, rats were subjected to forced treadmill running three times per week for the entire three month experimental period. The effect of this rigorous work regimen on the rats' heart was assessed using different parameters. The work capacity of each rat was tested in a forced swimming test, in which the length of time that the rat could swim with a weight attached to its tail. The weight was adjusted to approximately seven percent of the rat's body weight. The swimming tests were administered at the start of the experiment, and, at the beginning and end of the third and last month of the experiment.

The control Group I, comprised 15 rats that were not subjected to the rigorous treadmill exercise, and were not administered either of inosine or orotic acid. The rats in Group II comprised 15 animals that were subjected to the rigorous treadmill exercise regimen, but did not receive any supplement containing inosine and/or orotic acid. Group III were also subjected to the rigorous treadmill exercise regimen, but in this case, the animals were administered, through an intragastric tube, a composition of potassium orotate plus inosine comprising 100 mg/kg and 25 mg/kg, respectively, each day from the end of the second month until the end of the third and final month of the study. At the end of the second month the rats in both of Groups II and III developed clear signs of myocardial dystrophy due to overtraining. These signs included alteration in EKGs, decrease in body mass and a reduction of running time during the treadmill periods.

The parameters observed in all three groups were: EKG, working capacity (time of running endurance and swimming endurance), growth of body mass, oxygen consumption, and at the end of the experiment, anatomical and histological examination and electron microscopic examination of hearts. The level of physical exercise for Groups II and III during the experiment was not changed throughout the 3 month experimental period.

At the start of the experiment, all 50 rats swam for approximately 56 minutes±8 minutes. By the end of the second month of treadmill training of the animals in Groups II and III, the three groups showed the following swimming times until the point of exhaustion: Group II 38±4.2 minutes, Group III 35±4.0 minutes, and the unstressed and untreated control Group I 52±3.6 minutes. At this point the difference between Groups II and III were not statistically significant At the end of the third month of the experiment wherein Groups II and III continued the forced treadmill training, and Group III additionally received the potassium orotate plus inosine combination At the end of the experiment, the times of swimming to the point of exhaustion in the three groups were; Group I 50±4.0, Group II 17±4.5 and Group III 48±12 minutes, respectively (see Table 4-A).

TABLE 4-A

| | | | Work Capacity | | |
|---|---|---|---|---|---|
| | | | (in min of swimming) | | Relative Heart |
| Group | Animal | No of | After 2 months | After 3 months | Weight (mg/100 gr |
| No | Group | Animals | of training | of training | body weight) |
| I | Control | 15 | 52.4 ± 3.6 | 50.3 ± 4.2 | 29.2 ± 5.0 |
| | Animals | | | | |
| | (No training) | | | | |
| II | Control | 20 | 38.0 ± 4.2 | 16.8 ± 4.5 | 428 ± 16.0 |
| | animals. | | <0.01 | <0.001 | <0.05 |
| | Animals with | | | | |
| | myocardial | | | | |
| | dystrophy. | | | | |
| III | Animal with | 15 | 35.0 ± 3.6 | 48.0 ± 12.2 | 341 ± 3.0 |
| | myocardial dystrophy | | <0.01 | >0.1 | <0.05 |
| | received | | <0.001 | <0.05 | <0.05 |
| | inosine 25 mg/kg/BW | | | | |
| | and potassium | | | | |
| | orotate 100 mg/kg/BW | | | | |

LEGEND:

| | |
|---|---|
| P¹ | Statistical probability of difference from Group I |
| P² | Statistical probability of difference from Group II |
| BW | Body Weight |

The Group III animals received the inosine/potassium orotate treatment during the third
month of the experiment

The differences between Group I (control, no forced exercise/luntreated) and Group III (forced exercise and treated with potassium orotate and inosine) were not statistically significant, whereas the performance of the Group II (forced exercise and untreated) rats continuously deteriorated as measured by the significantly lower significantly swim time, approximately one third of the time in Groups I or III. The data clearly indicate the restorative effects of administering inosine/orotate in combination on the work capacity of the animals in Group III. Further, the restoration was virtually complete in that the swimming times were not statistically significant from the control Group I.

The heart related effects of overtraining were also reflected by the changes in EKG parameters and a dramatic drop in the body mass of rats. The EKG characteristics in Group II lacked positive dynamics and were the opposite of Group III where the EKG demonstrated a return to normal with no lasting negative alterations in the EKG observed (see Tables 4-B and 4-C).

TABLE 4-B

| Animal | Heart Rate (beats/min) | | | Systolic Index (%) | | |
|---|---|---|---|---|---|---|
| Group | | AFTER | | Initial | After | |
| Numbers | Initial Value | 2 Months | 3 Months | Value | 2 Months | 3 Months |
| I | | 420 ± 4.6 | 380 ± 13.4 | | 43.4 ± 6.7 | 41.2 ± 8.3 |
| P¹ | 400 ± 16.4 | >0.05 | >0.05 | 40 ± 13.4 | >0.05 | >0.05 |
| II | | 498 ± 6.4 | 500 ± 20.1 | | 66.0 ± 2.7 | 69.7 ± 3.1 |
| P¹ | 400 ± 16.4 | <0.001 | <0.001 | 40 ± 13.4 | <0.01 | <0.01 |
| III | | 494 ± 5.6 | 418 ± 18.3 | | 73.0 ± 3.1 | 42.2 ± 3.4 |
| P¹ | | <0.001 | >0.1 | | <0.001 | >0.9 |
| P² | | <0.001 | <0.01 | | >0.1 | <0.001 |

LEGEND:

| | |
|---|---|
| Group I | 15 rats; intact, no training |
| Group II | 20 Overtrained rats with myocardial dystrophy |
| Group III | 15 Overtrained rats with myocardial dystrophy given Potassium Orotate & |
| | Inosine |
| P¹ | Statistical probability of difference from Group I |
| P² | Statistical probability of difference from Group II |

TABLE 4-C

| Animal | PQ | | | QRS Complex | | |
|---|---|---|---|---|---|---|
| Group | | AFTER | | | After | |
| Numbers | Initial Value | 2 Months | 3 Months | Initial Value | 2 Months | 3 Months |
| I | | 0.057 ± 0.003 | 0.06 ± 0.003 | | 0.02 ± 0.002 | 0.02 ± 0.003 |
| P¹ | 0.06 ± 0.003 | >0.05 | | 0.02 ± 0.004 | | |
| II | | 0.05 ± 0.004 | 0.043 ± 0.003 | | 0.020 ± 0.002 | 0.03 ± 0.003 |
| P¹ | 0.06 ± 0.003 | >0.2 | <0.001 | 0.02 ± 0.004 | <0.05 | <0.02 |
| III | | 0.052 ± 0.002 | 0.06 ± 0.006 | | 0.025 ± 0.003 | 0.02 ± 0.002 |
| P¹ | | >0.2 | >0.7 | | >0.05 | — |
| P² | | >0.1 | | | >0.6 | <0.02 |

| Animal | QT | | | R (B mv) | | |
|---|---|---|---|---|---|---|
| Group | | AFTER | | | AFTER | |
| Numbers | Initial Value | 2 Months | 3 Months | Initial Value | 2 Months | 3 Months |
| I | | 0.06 ± 0.002 | 0.062 ± 0.003 | | 0.57 ± 0.06 | 0.6 ± 0.04 |
| P¹ | 0.058 ± 01.006 | >0.05 | >0.05 | 0.47 ± 0.06 | >0.05 | >0.05 |
| II | | 0.052 ± 0.003 | 0.086 ± 0.003 | | 0.26 ± 0.03 | 0.18 ± 0.03 |
| P¹ | 0.058 ± 01.006 | <0.001 | <0.01 | 0.47 ± 0.06 | <0.001 | <0.001 |
| III | | 0.06 ± 0.002 | 0.065 ± 0.002 | | 0.3 ± 0.06 | 0.69 ± 0.03 |
| P¹ | | — | >0.4 | | <0.01 | >0.05 |
| P² | | <0.001 | <0.001 | | >0.6 | <0.001 |

LEGEND:

| | |
|---|---|
| Group I | 15 rats; intact, no training |
| Group II | 20 Overtrained rats with myocardial dystrophy |
| Group III | 15 Overtrained rats with myocardial dystrophy given Potassium Orotate & Inosine |
| P¹ | Statistical probability of difference from Group I |
| P² | Statistical probability of difference from Group II |

Table 5 shows that an optimal regime of overtraining with inosine and potassium orotate treatment (Group III) led to positive shifts in the ion composition in the myocardium; there was a moderate decrease in potassium and a more marked one of sodium. In Group III there was no decrease in potassium, the sodium level dropped considerably, and this resulted in an increase in the potassium/sodium ratio.

TABLE 5

| Animal | LEVELS OF | | |
|---|---|---|---|
| Group | POTASSIUM | SODIUM | P/S |

| | | |
|---|---|---|
| Numbers | mcq/gram of dried myocardial tissue | Ratio |

| | | | |
|---|---|---|---|
| I | 0.362 ± 0.008 | 0.249 ± 0.011 | 1.45 ± 0.06 |
| II | 0.378 ± 0.014 | 0.279 ± 0.016 | 1.35 ± 0.05 |
| P¹ | >0.1 | >0.1 | >0.1 |
| III | 0.346 ± 0.015 | 0.188 ± 0.015 | 1.84 ± 0.19 |
| P¹ | >0.3 | <0.01 | >0.05 |
| P² | >0.05 | <0.001 | <0.05 |

LEGEND:
Group I 15 rats; intact, no training
Group II 20 Overtrained rats with myocardiodystrophy
Group III 15 Overtrained rats with myocardiodystrophy given Potassium Orotate & Inosine
P¹ Statistical probability of difference from Group I
P² Statistical probability of difference from Group II
P/S Ratio Potassium/Sodium ratio
mcq/gram microgram per gram

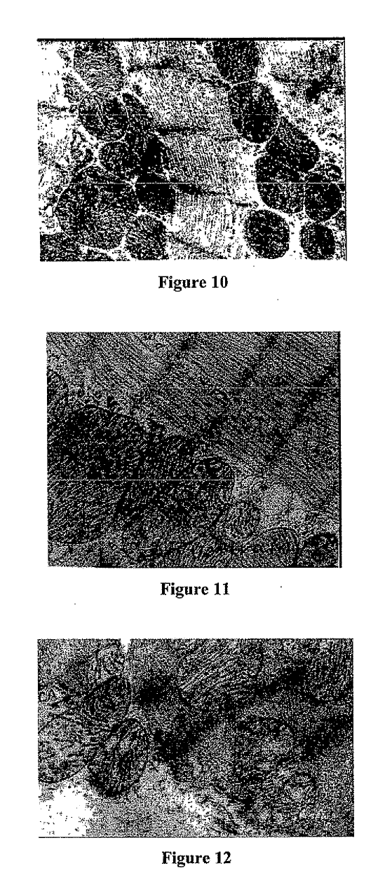

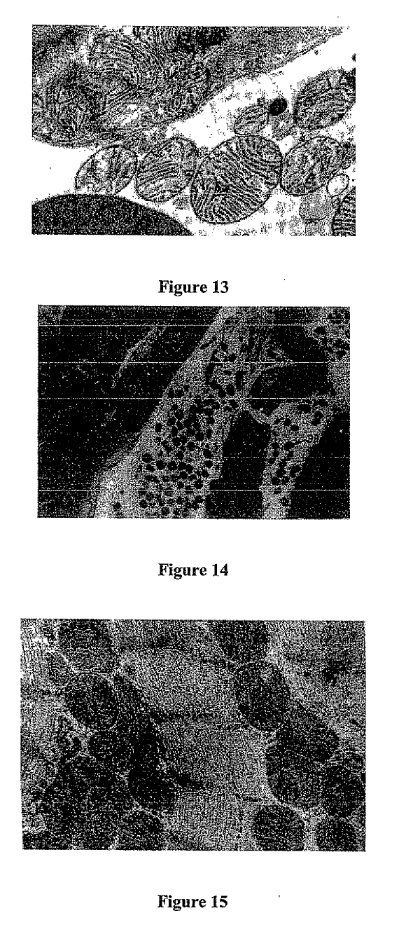

Rats in Group II showed an increase in the relative weights of the heart, lungs, liver and adrenals, and a decrease in the weight of the thymus and the thyroid gland. Potassium orotate plus inosine (Group III) restored the relative weights of these organs (Table 5). The myocardium of rats in Group III showed a slight thickening of the muscle fibers with no other histological alterations. In Group II, there was a widening of intramuscular spaces filled with lymphocytes, and blood providing evidence of hemorrhages, and the cytoplasm of muscle cells stained poorly with eosin. No such negative findings were noted in the myocardial tissues of rats in Group III. Ultrastractural studies of the myocardium of the Group II rats revealed foci of ruptured sarcolemma, presence of mitochondria in intercellular spaces and widening of cisterns in the sarcoplasma reticulum, which indicate structurally severe compromising of myocardial cells. In contrast, such structural alterations were not observed in Group III. See FIGS. 10 through 15.

It is further noteworthy that overtraining also increased the relative mass of skeletal muscles in Groups III, but not in Group II (not shown).

In conclusion, overtraining negatively affected various functional and structural characteristics of the hearts of animals, which expressed itself by a decrease in working capacity and body mass, aberrations in the EKG and morphological evidence that document the development of a deteriorated myocardium. The combination of orotic acid, as potassium orotate, in combination with inosine substantially reduced or eliminated the negative functional and structural alterations otherwise caused by excessive physical exercise or overtraining.

### Example 14

### Work Capacity

Studies on the effect of the combination of inosine and orotate were premised upon the belief that a pre-condition for improving one's athletic performance, i.e., work capacity, is increasing the intensity and quantity of training. In this coniection, athletic training has recently been supplemented with various pharmacologic agents that are believed capable of increasing working capacity. However, it is further appreciated that administering these substances should not endanger the health of the athletes. Certain compounds among pharmacologic agents, capable of regulating motor activity in humans, are orotic acid and its salts. Orotate is a direct precursor of the pyrimidine bases of nucleic acids. Orotic acid administration stimulates the biosynthesis of proteins and enzymes and is involved in several aspects of cell metabolism. Similarly, there is a growing interest in studying purine derivatives, particularly inosine, participating in the formation of nucleic acids, proteins and energy-supplying substrates (glycogen, AMP, ADP and ATP). The objective of the study was to examine whether the application of inosine or orotate individually or in combination could contribute to an increase in the working capacity of animals.

### A. Example 14(a)

### Short Term in Vivo Evaluation of Inosine and Orotate in Mice

Seventy two animals (mice) were divided into 12 groups of 6 animals each; a control group (untreated) and 11 test groups that were intragastrically administered varying daily doses of inosine, potassium orotate or an inosine plus potassium orotate combination (the “combination”) for 14 days. The animals were observed at rest and immediately after a forced swimming exercise on day 14 of the experiment. The swimming exercise consisted of forcing the animals to perform repetitive swimming in a three meter long channel until exhaustion. The level of exhaustion was determined by a count of the number of swimming lengths which each animal did within 30 minutes. A lower number of swimming lengths indicated a greater level of exhaustion, whereas a higher number of lengths indicated a greater work capacity.

Inosine was administered intragastrically in doses of 12.5, 25, 50 and 100 mg/kglbody weight daily for 14 days. Administering inosine individually increased the number of swimming lengths traversed within the 30 minute time limit by 35.4%, 85.6%, 58.5% and 24.3%, respectively; whereas potassium orotate, in doses of 25, 50, 100 and 200 mg/kg/body weight a day for 14 days given intragastrically, produced mean increases in swimming lengths traversed of 60.7%, 82.2%, 125% and 78.3%, respectively. Thus, 25 mg/kg body weight of inosine or 50 mg/kg/body weight of potassium orotate were the most effective doses. (See Table 6).

Experiments with various combinations of both the agents showed that all of their combinations were synergistic, exceeding effects of either component given alone. The combination of 25 mg/kg/body weight inosine combined with 50 mg/kg/body weight potassium orotate per day for 14 days, proved to be most effective with an increase of the swimmng lengths traversed of 165%, which is more than 30% greater than the greatest effects of either inosine or potassium orotate alone.

TABLE 6

| | | Potassium | |
|---|---|---|---|
| | Inosine | Orotate | |
| | Swimming | Swimming | Inosine plus Orotate |
| Dosage | lengths | lengths | Swimming lengths |
| Control, no treatment | 24.9 | 24.9 | 24.9 |
| 12.5 mg/kg BW | 33.9 | | |
| 25 mg/kg BW | 46.4 | 40.2 | |
| 50 mg/kg BW | 39.6 | 45.6 | |
| 100 mg/kg BW | 31.1 | 56.3 | |
| 200 mg/kg BW | | 44.6 | |
| 25 mg/kg + 25 mg/kg BW | | | 57.5 |
| 25 mg/kg + 50 mg/kg BW | | | 66.4 |
| 25 mg/kg + 100 mg/kg BW | | | 60.8 |

(BW = Body Weight)

Biochemical analysis of the glycogen content in the liver of the inosine (25 mg/kg), potassium orotate (50 mg/kg), and the combination fed animals demonstrated an increased hepatic glycogen content by 21%, 18% and 40%, respectively, which suggests that the enhanced work capacity is due to an enhancement of the animal's energy producing potential. The animal swimming exercise decreased hepatic glycogen stores by 67.5% from the baseline in the control animals, whereas inosine, potassium orotate or their combination significantly slowed hepatic glycogen expenditure during the exercise. The mean decreases being significantly (P<0.05) less than in the controls—only 24.5%, 18.5% and 27.5%, respectively. The shrinking of the liver glycogen stores was further associated with a significant diminution of blood lactic acid.

### B. Example 14(b)

### Long-Term (6 Months) In Vivo Experiments on Rats

Seventy two white rats were randomly placed in 6 groups (12 animals each), to test the effectiveness of training and to determine morphological and physiological alterations in the rats. In Group I, control rats were nontrained and not administered either of inosine or the combination of inosine and orotate; Group II comprised non-trained rats to which were administered inosine (25 mg/kg/body weight; Group III comprised non-trained rats to which were administered the combination of inosine plus potassium orotate (25 mg/kg/body weight of inosine and 50 mg/kg/body weight potassium orotate); Group IV comprised trained rats that were not administered either of inosine or the combination of inosine and orotate; Group V comprised trained rats to which was administered inosine alone (25 mg/kg/body weight), and Group VI comprised trained rats to which was administered the combination of inosine plus potassium orotate (25 mg/kg/body weight of inosine and 50 mg/kg/body weight potassium orotate).

Training of rats consisted of their forced swimming in a water bath (28-30° C.) with a weight fixed on their backs (7.5% of the rat body weight). During the first two weeks the animals swam daily for 5-10 minutes, after which we determined the individual maximum time of swimming to exhaustion (i.e. maximum work capacity) for each animal and then calculated the mean maximum time for each group. After 2 weeks these initial maximum work capacity values were used for planning and performing additional swimming training.

The functional condition of the rats at rest was estimated from their work capacity (maximum swimming time), body weight increase, EKG tests, oxygen consumption and sodium and potassium levels in the myocardium in a relatively quiet state. The morphological state of the heart and other viscera were rated from their absolute and relative weights, and also from histological and histochemical examinations of the myocardium. Also determined were the amounts of glycogen in the m. quadriceps (quadriceps group of muscles) of the hip, the myocardium and the liver. All the quantitative results obtained were processed with conventional statistical procedures.

At the start of the study, the initial work capacity (the maximum swimming time sustained) of rats in all the groups in the study was approximately the same and equaled 65-77 minutes on an average. The work capacity of non-trained and non-medicated animals after 6 months of observation (Group I) did not significantly differ from the initial values. The working capacity of the Group IV rats (i.e., trained but not administered either inosine or the combination of inosine/orotate) was significantly greater than at the beginning, i.e., approximately 142±2.4 minutes. The greatest increase seen after 6 months of training was in those animals receiving potassium orotate plus inosine (Group VI) with a maximum mean swimming time of 212±4.2 minutes. See Table 7-A.

TABLE 7A

| Group | | | Dose | Body Weight | | Swimming time - MIN. | | Heart weight after 6 mo. |
|---|---|---|---|---|---|---|---|---|
| No | NN | Product | mg/kg | Starting | After 6 mo. | Before | After 6 mo. | mg/100gk BW |
| I | 12 | Contr | — | 120 ± 5.5 | 275 ± 68 | 62 ± 6.0 | 60 ± 5.0 | 292 ± 4.0 |
| II | 12 | INO | 25 | 133 ± 6.1 | 280 ± 3.8 | 65 ± 5.0 | 70 ± 4.0 | 302 ± 91 |
| | P¹ | NS | | | NS | | NS | NS |
| III | 12 | PO | 50 | 115 ± 6.0 | 286 ± 8.5 | 72 ± 4.0 | 80 ± 4.2 | 304 ± 9.2 |
| | P¹ | NS | | NS | NS | NS | P < 0.05 | NS |
| IV | 12 | Contr | — | 120 ± 3.0 | 289 ± 5.8 | 59 ± 5.0 | 142 ± 2.4 | 325 ± 2.8 |
| | P¹ | | | NS | NS | NS | <0.001 | <0.05 |
| V | 12 | INO | 25 | 121 ± 4.5 | 290 ± 7.4 | 65 ± 6.6 | 180 ± 3.4 | 359 ± 3.9 |
| | P¹ | | | NS | <0.05 | NS | <0.001 | <0.001 |
| | P² | | | NS | NS | NS | <0.05 | <0.05 |
| VI | 12 | INO + PO | 25/50 | 120 ± 6.1 | 295 ± 8.8 | 59 ± 7.0 | 212 ± 6.0 | 399 ± 4.2 |
| | P¹ | | | NS | <0.05 | NS | <0.001 | <0.001 |
| | P² | | | NS | NS | NS | <0.001 | <0.05 |
| | P³ | | | NS | NS | NS | <0.05 | <0.05 |

LEGEND:

| | |
|---|---|
| P¹ | Statistical probability of difference from Group I |
| P² | Statistical probability of difference from Group II |
| P³ | Statistical probability of difference from Group III |
| INO | Inosine |
| PO | Potassium Orotate |
| NN | Number of Animals |
| BW | Body Weight |
| NS | Not Significant |
| Group I | Control-Untrained |
| Group II | Untrained-Inosine |
| Group III | Untrained Inosine + Potassium Orotate |
| Group IV | Control-Trained |
| Group V | Trained-Inosine |
| Group VI | Trained-Inosine + Potassium Orotate |

In this experiment, the animals of Group I, II, and III were in normal (untrained), motor activity Groups IV, V
and VI underwent physical training
The animals of Groups I and IV were controls (no medication).
Animals of Groups II and V received inosine 25 mg/kg/BW
Groups III and VI received a daily admixture of inosine 25 mg/kg/BW and potassium orotate 50 mg/kg/BW

Body weights of non-trained rats after 6 months were 275-288 grams. Body weights of animals, which trained (Groups IV, V, and VI) were 289-295 g after 6 months: thus, the administration of inosine or inosine plus potassium orotate did not materially alter animal body weights (see Table 7-A).

Similarly, inosine or inosine plus potassium orotate did not significantly influence values of the specific oxygen consumption by the non-trained rats. Whereas, in rats trained for 6 months, this parameter decreased (improved) by 25% compared to the trained non-medicated controls. In the rats given inosine plus potassium orotate the decrease was 36%, a 44% improvement over the non-treated rats (see Table 7-B).

Quantitative histochemical and biochemical determinations of glycogen in the myocardium, liver and m. quadriceps femoris demonstrated the greatest concentrations in trained animals given inosine and potassium orotate, and that the medication brought about more economical utilization of gycogen in animals which underwent the physical training. (Table 7-B)

TABLE 7-B

| | | | | Heart weight | Oxygen consumption | Glycogen in m. quadriceps | |
|---|---|---|---|---|---|---|---|
| Group | | | Dose | after 6 mo. | ml/min/kg BW | End of experiments. | |
| No | NN | Product | mg/kg | mg/100gk BW | End of experiment | In rest | After work |
| I | 12 | Contr | — | 292 ± 4.0 | 28.0 ± 0.72 | 661 ± 21.0 | 240 ± 47.5 |
| II | 12 | INO | 25 | 302 ± 91 | 26.0 ± 0.82 | 829 ± 42.0 | 450 ± 28.3 |
| | P¹ | NS | | NS | <0.05 | <0.05 | <0.05 |
| III | 12 | PO | 50 | 304 ± 9.2 | 27.0 ± 0.60 | 860 ± 16.0 | 488 ± 15.0 |
| | P¹ | NS | | NS | <0.05 | <0.05 | <0.05 |
| IV | 12 | Contr | — | 325 ± 2.8 | 21.0 ± 0.55 | 760 ± 18.0 | 383 ± 24.8 |
| | P¹ | | | <0.05 | <0.05 | <0.05 | <0.05 |
| V | 12 | INO | 25 | 359 ± 3.9 | 220 ± 0.049 | 889 ± 26.0 | 558 ± 13.4 |
| | P¹ | | | <0.001 | <0.05 | <0.05 | <0.05 |
| | P² | | | <0.05 | NS | <0.05 | <0.05 |
| VI | 12 | INO + PO | 25/50 | 399 ± 4.2 | 180 ± 0.61 | 1064 ± 24.0 | 729 ± 24.8 |
| | P¹ | | | <0.001 | <0.05 | <0.001 | <0.001 |
| | P² | | | <0.05 | <0.05 | <0.05 | <0.05 |
| | P³ | | | <0.05 | <0.05 | <0.05 | <0.001 |

LEGEND:

| | |
|---|---|
| P¹ | Statistical probability of difference from Group I |
| P² | Statistical probability of difference from Group II |
| P³ | Statistical probability of difference from Group III |
| INO | Inosine |
| PO | Potassium Orotate |
| NN | Number of Animals |
| BW | Body Weight |
| NS | Not Significant |
| Group I | Control-Untrained |
| Group II | Untrained-Inosine |
| Group III | Untrained Inosine + Potassium Orotate |
| Group IV | Control-Trained |
| Group V | Trained-Inosine |
| Group VI | Trained-Inosine + Potassium Orotate |

In this experiment, the animals of Group I, II, and III were in normal (untrained) motor activity Groups IV, V and
VI underwent physical training
The animals of Groups I and IV were controls (no medication).
Animals of Groups II and V received inosine 25 mg/kg/BW
Groups III and VI received a daily admixture of inosine 25 mg/kg/BW and potassium orotate 50 mg/kg/BW

Further, neither inosine nor inosine plus potassium orotate altered EKGs in non-trained rats (See Table 7-C). 6-month-long trainings without the medication caused slowing of the heart rate, and lengthening of the QT interval. In trained rats inosine, and more so, inosine plus potassium orotate, resulted in greater degrees of cardiac slowing and an increase in the sum height of the R waves on the EKG (see Table 7-C).

TABLE 7-C

| | | | AFTER 6 MONTHS | | | | | |
|---|---|---|---|---|---|---|---|---|
| | | | | | | TRAINING | | |
| | | | NORMAL MOTOR ACTIVITY-UNTRAINED | | | | | Inosine + |
| Param- | | | | | Inosine + | | | Potassium |
| eters | | | Control | Inosine | Potassium | Control | Inosine | Orotate |
| of ECG | | Initial Value | (Group I) | (Group II) | Orotate (Group III) | (Group IV) | (Group V) | (Group VI) |
| HEART | | 480 ± 9.2 | 468 ± 8.4 | 452 ± 8.6 | 468 ± 8.2 | 350 ± 4.6 | 300 ± 5.2 | 296 ± 6.41 |
| RATE | P¹ | | >0.05 | >0.05 | >0.3 | <0.001 | <0.001 | <0.001 |
| | P² | | | | | | <0.001 | <0.001 |
| | P³ | | | | | | | >0.5 |
| PQ | | 0.048 ± 0.004 | 0.043 ± 0.002 | 0.054 ± 0.003 | 0.06 ± 0.088 | 0.046 ± 0.001 | 0.047 ± 0.03 | 0.05 ± 0.002 |
| | P¹ | | >0.3 | >0.3 | >0.2 | >0.7 | >0.8 | >0.7 |
| QRS | | 0.03 ± 0.004 | 0.028 ± 0.005 | 0.026 ± 0.003 | 0.03 ± 0.005 | 0.03 ± 0.009 | 0.031 ± 0.004 | 0.029 ± 0.006 |
| | P¹ | | >0.7 | >0.5 | | | >0.8 | >0.9 |
| QT | | 0.058 ± 0.006 | 0.06 ± 0.008 | 0.06 ± 0.001 | 0.062 ± 0.003 | 0.068 ± 0.001 | 0.076 ± 0.001 | 0.074 ± 0.002 |
| | P¹ | | >0.9 | >0.9 | >0.5 | >0.05 | <0.01 | <0.01 |
| | P² | | | | | | <0.001 | <0.001 |
| ΣR (mm) | | 13 ± 1.2 | 13.8 ± 0.8 | 14 ± 0.7 | 14.2 ± 0.5 | 15 ± 0.4 | 17 ± 0.5 | 21 ± 0.3 |
| | P¹ | | >0.05 | >0.8 | >0.6 | | <0.05 | <0.02 |
| | P² | | | | | | <0.02 | <0.001 |
| | P³ | | | | | | | <0.001 |
| Systolic | | 46 ± 9.4 | 43 ± 3.7 | 42.5 ± 9.7 | 47 ± 4.5 | 40 ± 2.4 | 38 ± 6.3 | 35 ± 2.4 |
| Index | P¹ | | >0.7 | >0.6 | >0.8 | >0.6 | >0.4 | >0.2 |
| | P² | | | | | | >0.7 | >0.2 |
| | P³ | | | | | | | >0.4 |

LEGEND:

| | |
|---|---|
| P¹ | Statistical probability of difference from Initial Value |
| P² | Statistical probability of difference from Group IV |
| P³ | Statistical probability of difference from Group V |

Heart Rate-beats per minute
PQ, QRS, QT, ΣR(mm), Systolic Index-Standard EKG (electrocardiogram) measurement designations

| | |
|---|---|
| Group I | Control-Untrained 12 animals |
| Group II | Untrained-Inosine 12 animals |
| Group III | Untrained Inosine + Potassium Orotate 12 animals |
| Group IV | Control-Trained 12 animals |
| Group V | Trained-Inosine 12 animals |
| Group VI | Trained-Inosine + Potassium Orotate 12 animals |

The compounds studied failed to alter the relative weights of viscera in nontrained rats, whereas the trained rats exhibited increased relative weights of the heart, lungs, liver, kidneys and adrenal glands (Table 7-D). In trained rats given inosine, and more so for those given the inosine plus potassium orotate, for 6 months, the relative weights of the heart, kidneys and adrenal glands were significantly higher than in the non-medicated trained rats, as shown below.

TABLE 7-D

| Animal | ORGAN WEIGHT (mg) PER 100 GRAMs Body Weight | | | | | | | |
|---|---|---|---|---|---|---|---|---|
| Group | | Thyroid | | | | Kidneys | Adrenals | |
| No | Thymus | Gland | Lungs | Heart | Liver | (both) | (both) | Spleen |
| I | 142.6 ± 5.6 | 8.8 ± 0.3 | 551 ± 22 | 292.2 ± 4.0 | 3086 ± 184 | 669.6 ± 15.6 | 23.9 ± 0.7 | 275 ± 3.7 |
| II | 135.4 ± 5.8 | 8.6 ± 0.8 | 542 ± 62.1 | 309.4 ± 9.7 | 3080 ± 193 | 683.7 ± 1.2 | 25.2 ± 1.2 | 278.6 ± 8.7 |
| P¹ | >0.6 | >0.8 | >0.9 | >0.2 | >0.8 | >0.8 | >0.4 | >0.9 |
| III | 140.4 ± 7.6 | 8.5 ± 0.4 | 560 ± 42.1 | 304.6 ± 9.2 | 3070 ± 189 | 712 ± 15.6 | 24.3 ± 0.6 | 278 ± 9.6 |
| P¹ | >0.7 | >0.6 | >0.8 | >0.2 | >0.9 | >0.7 | >0.4 | >0.9 |
| IV | 103.1 ± 9.1 | 9.6 ± 0.2 | 861 ± 36.2 | 325.2 ± 2.8 | 3433 ± 80.1 | 762 ± 14.2 | 29.3 ± 1.3 | 256 ± 14.3 |
| P¹ | <0.001 | <0.001 | <0.001 | <0.001 | >0.05 | <0.001 | <0.01 | >0.2 |
| V | 126.2 ± 5.8 | 9.4 ± 0.4 | 902.2 ± 59.2 | 359.3 ± 4.5 | 2915 ± 155 | 805.7 ± 32 | 30.5 ± 1.3 | 254 ± 10.1 |
| P¹ | >0.05 | >0.3 | <0.001 | <0.001 | >0.5 | <0.01 | <0.001 | >0.2 |
| P² | <0.05 | >0.7 | >0.6 | <0.01 | <0.01 | <0.02 | >0.5 | >0.9 |
| VI | 148.6 ± 9.6 | 9.2 ± 0.3 | 941.6 ± 33.2 | 399.4 ± 4.2 | 3018 ± 11.6 | 858.6 ± 25 | 30.7 ± 0.9 | 271.2 ± 9.6 |
| P¹ | >0.5 | >0.4 | <0.001 | <0.001 | >0.6 | <0.001 | <0.001 | >0.7 |
| P² | <0.001 | >0.2 | <0.001 | <0.001 | >0.7 | <0.001 | >0.4 | >0.2 |
| P³ | >0.05 | >0.7 | >0.2 | <0.001 | >0.4 | <0.001 | >0.9 | >0.3 |

LEGEND:

| | |
|---|---|
| P¹ | Statistical probability of difference from Group I |
| P² | Statistical probability of difference from Group IV |
| P³ | Statistical probability of difference from Group V |
| Group I | Control-Untrained 12 animals |
| Group II | Untrained-Inosine 12 animals |
| Group III | Untrained Inosine + Potassium Orotate 12 animals |
| Group IV | Control-Trained 12 animals |
| Group V | Trained-Inosine 12 animals |
| Group VI | Trained-Inosine + Potassium Orotate 12 animals |

The electrolyte studies in the myocardium showed that inosine promoted an increase in the level of potassium, and inosine plus potassium orotate significantly decreased the myocardial sodium level (Table 7-E). These and other measured parameters of electrolyte metabolism indicate positive trends in the electrolyte composition in the cardiac wall of trained rats.

TABLE 7-E

| | | | | | | P/S | | | |
|---|---|---|---|---|---|---|---|---|---|
| Animal | Potassium | | Sodium | | P/S | In rat sacrificed | | | |
| Group | mcq/gram | | mcq/gram | | In all rats | In quiet | | after intense | |
| No | dry tissue | # | dry tissue | # | group | state | # | physical exercise | # |
| I | 0.351 ± 0.01 | (12) | 0.312 ± 0.02 | (12) | 1.12 ± 0.1 | 1.34 ± 0.2 | (6) | 1.09 ± 0.1 | (6) |
| II | 0481 ± 0.03 | (12) | 0.261 ± 0.018 | (12) | 1.85 ± 0.05 | 2.01 ± 0.3 | (6) | 1.67 ± 0.09 | (6) |
| P¹ | <0.02 | | >0.05 | | <0.001 | >0.05 | | <0.001 | |
| III | 0.372 ± 0.003 | (12) | 0.228 ± 0.02 | (12) | 1.63 ± 0.2 | 1.7 ± 0.13 | (6) | 1.49 ± 0.17 | (6) |
| P¹ | >0.2 | | <0.01 | | <0.02 | >0.2 | | >0.05 | |
| IV | 0.364 ± 0.01 | (12) | 0.259 ± 0.017 | (12) | 1.4 ± 0.14 | 1.12 ± 0.06 | (6) | 1.64 ± 0.2 | (6) |
| P¹ | >0.4 | | >0.05 | | >0.1 | >0.4 | | <0.05 | |
| V | 0408 ± 0.015 | (12) | 0.304 ± 0.018 | (12) | 1.34 ± 0.12 | 1.16 ± 0.08 | (6) | 1.55 ± 0.04 | (6) |
| P¹ | <0.01 | | >0.7 | | >0.2 | >0.5 | | <0.001 | |
| P² | <0.05 | | >0.05 | | >0.7 | >0.09 | | >0.4 | |
| VI | 0.364 ± 0.015 | (12) | 0.275 ± 0.016 | (12) | 1.33 ± 0.04 | 1.22 ± 0.12 | (6) | 1.41 ± 0.07 | (6) |
| P¹ | >0.4 | | >0.05 | | <0.05 | >0.5 | | <0.001 | |
| P² | — | | >0.1 | | >0.4 | >0.6 | | >0.3 | |
| P³ | >0.05 | | >0.05 | | >0.9 | >0.7 | | >0.5 | |

LEGEND:

| | |
|---|---|
| P | Potassium |
| S | Sodium |
| # | Number in parenthesis indicate number of animals |
| mcq/gram | micrograms per gram |
| P¹ | Statistical probability of difference from Group I |
| P² | Statistical probability of difference from Group IV |
| P³ | Statistical probability of difference from Group V |
| Group I | Control-Untrained |
| Group II | Untrained-Inosine |
| Group III | Untrained Inosine + Potassium Orotate |
| Group IV | Control-Trained |
| Group V | Trained-Inosine |
| Group VI | Trained-Inosine + Potassium Orotate |

### Example 15

### Observations on Athletes

The results of the animal experiments described above, establishing the benefits of administering a combination of inosine and orotic acid, or a salt thereof, on enhancing work capacity was further extended to human athletes. In brief, the effectiveness of using a combination of inosine and potassium orotate in training of high-level human athletes was established.

Two groups of six human cyclists were analyzed. Group I was a control, non-treated group of cyclists, and Group II wherein the cyclists received a composition comprising 0.5 gram/day inosine plus 1 gram/day potassium orotate, for 4 weeks. Each group was submitted to three training sessions per week. Special cycling models were used with variable resistance capabilities that simulate either aerobic or anaerobic cycling conditions that are used for preparing for athletic competition. In brief, the study established the stimulatory effect of the composition on the athlete's work capacity, as manifested in significant improvements in both aerobic and anaerobic exercise. The mean increase in work capacity was approximately 17.9% for aerobic work and 17.5% for anaerobic work.

Measurements obtained during Group I's seventh training session demonstrated an increase in maximal work capacity of approximately 6.1% when compared to day 1 of the study. In contrast, measurements taken during Group II's ninth session demonstrated a maximal work capacity increase of 12.0% over the day 1 measurements. This amounts to a 96% increase over the 6.1% in the control Group I. A similar trend was seen with measurements of the PWC₁₇₀ (Physical Work Capacity at 170 heart beat/minute rate) test results.

The inosine and potassium orotate composition also induced certain beneficial shifts in EKG and polycardiographic indices, suggesting an increased cardiac functional reserve. For example, the cyclists in Group II were able to diminish their cardiac rate, prolong the QRST segment (by 9 msec), reduce dysaxia for the maximum vectors of the QRS and T wave angles, and enhanced the T wave amplitude sum in the standard EQG leads.

Measured against standard indices the results showed marked “economization” of the left cardiac ventricle activity in athletes that were administered the inosine+potassium orotate combination. The beneficial effects of the combination were seen from the following phase shifts in the polycardiograms: slowed cardiac rhythm, enhanced mechanic and total systole, prolonged ejection phase and electric systole, reduced cardiac minute volume ejection time and the actual systolic index. Thus, these shifts in cardiodynamics may be regarded as signs of an increased functional reserve in the “athletic” heart.

In addition to increasing capacity for muscular work, the inosine−potassium orotate composition maintained the blood nitrogen balance which is seen from stable blood urea levels during the entire experiment (42.8±4.5 to 45.1±8.9 mg/100 ml). The stabilizing influence on the anabolic-catabolic state of protein metabolism becomes more obvious when comparing these figures with the controls where blood urea rose progressively versus the average, from 43.2±4.2 to 58.3±4.1 mg/100 ml), i.e. it exceeded both the baseline level (by 35%) and the mean standard blood urea level (36-42 mg/100 ml).

The use of the inosine plus potassium orotate composition facilitated glycogen accumulation in peripheral white blood cells, and it is known that glycogen is an indispensable substrate for phosphocreatine and ATP biosynthesis in working skeletal muscle, and more of it is found in better trained athletes.

It is important to note that the beneficial influence of the inosine plus potassium orotate composition on white blood cell glycogen amount in athletes was accompanied by a statistically significant decrease in glycogen utilization during exercise. Taking into account that diminished carbohydrate synthesis and a drop in peripheral white blood cell glycogen levels are seen in athletes during exhausting muscular exertion, one may conclude that administration of the inosine−potassium orotate composition was highly effective in normalizing the body's energy balance, e.g., restoring normal glycogen and ATP levels.

Enhanced glycogen accumulation by body tissues, including peripheral white blood cells, was ascertained in cyclists taking larger doses of a composition of inosine plus potassium orotate (1.0 gram plus 3.0 gram, respectively, per day) for 7 days. This energy reserve is likely to be used by the body to increase its anaerobic work capacity, which was reflected in a markedly increased glycolytic activity, i.e., rise in blood lactic acid levels at 3 minutes of rehabilitation period after muscular exercise. Simultaneously, there was an activation of oxidation-reduction processes in the body as judged from significantly increased blood levels of newly formed lactic acid.

In conclusion, the studies with mice establish that administering a combination of inosine (intragastrically, 12.5, 25.0, 50 and 100 mg/kg body weight/day) and potassium orotate (25, 50, 100 and 200 mg/kg body weight/day) and their composition in doses, respectively, 25+25, 25+50, 25+100 mg/kg body weight, significantly increased the number of swimming lengths (i.e., distance) in white mice. These agents significantly increased hepatic glycogen stores and produced “economic” utilization at exercise. The effects were greatest with the 25 mg inosine+50 mg potassium orotate composition.

In addition, the studies with rats indicate that long term daily administration of inosine alone (25 mg/kg body weight) or an inosine (25 mg/kg body weight plus potassium orotate (50 mg/kg body weight) composition in rats undergoing training contributed to dramatic increases in work capacity, relative skeletal muscle weights and the weights of most viscera. The combination also affected an increase in glycogen stores in the myocardium, liver and skeletal muscles. Inosine, and more so, the inosine plus potassium orotate composition also decreased oxygen consumption and heart rate in a quiet state and positively influenced the composition and shifts in myocardial electrolytes in trained animals, indicating a more economic metabolic state had been achieved.

An additional benefit is that in both non-trained and trained rats, long term administration of inosine, alone or combined with potassium orotate, did not impede the growth of body weight and did not manifest any toxic effects on the functional state of the animals in general, including the morphology of the myocardium.

The studies of human cyclists showed that the combination of inosine and orotate produced an increase in their work capacity for strenuous muscular activity that reached its maximum by the end of the third week of the training course. This increase was significantly greater than that observed in the untreated group. The combination also increased their general capacity for muscular work as measured in the PWC₁₇₀ test.

The inosine plus potassium orotate composition induced enhancements of muscular capacity in athletes. These enhancements were associated with increased glycogen content in peripheral white blood cells and an enhanced functional energy reserve of the heart as indicated by improving indices of cardiac bioelectric activity and myocardial contractility. Higher doses of the inosine and potassium orotate composition, 1.0 and 3.0-g/day for 7 days, respectively, facilitated glycogen deposition in the tissues, which provided an increase in both energy potential and anaerobic resources in an athlete's body.

The foregoing studies indicate that administering a composition comprising both inosine and orotic acid, more typically in the form of an orotate salt comprising an inorganic cation, confers a surprising level of benefits to cardiac health and performance in mammals, including human patients and subjects. Persons of ordinary skill in the art will readily appreciate that the exemplifled embodiments of the compositions and the methods of use disclosed herein, are for illustrative purposes, and do not limit the scope of the methods and compositions of the current invention. Accordingly, persons of ordinary skill in the art will readily appreciate that there are additional embodiments of the compositions, methods of use, dosing regimen and the like, encompassed by the disclosure herein, as well as the claimed subject matter.

What is claimed is:

1. A composition comprising an amount of inosine or salt or ester thereof, and an amount of orotic acid or acylating derivative thereof or salt thereof and a pharmaceutically acceptable carrier thereof,
wherein said inosine or salt or ester thereof and orotic acid or acylating derivative or salt thereof, are present in an amount effective to treat a medical condition of the heart in a mammal suffering from same, said salt of orotic acid comprising a cation associated with the orotate, and said cation excluding lysine.

2. The composition of claim 1 wherein the amount effective to treat a medical condition of the heart in a mammal provides a synergistic therapeutic effect.

3. The composition of claim 1 wherein the composition is free of an amino acid salt of orotic acid.

4. The composition according to claim 1 wherein the composition comprises inosine and orotic acid or an inorganic salt of orotic acid.

5. The composition of claim 1, wherein the weight ratio of inosine or ester thereof or salt thereof to orotic acid, or acylating derivative or salt thereof, is from approximately 1:10 to approximately 10:1.

6. The composition of claim 5, wherein the weight ratio ranges from about 1:4 to about 4:1.

7. The composition of claim 1, wherein the calculated weight ratio of the inosine to the orotic acid ranges from about 1:10 to about 10:1.

8. The composition of claim 7, wherein the calculated weight ratio ranges from inosine about 1:4 to about 4:1.

9. The composition of claim 4, wherein the inorganic salt thereof comprises at least one inorganic monovalent or divalent metal cation or trivalent metal cation.

10. The composition of claim 9, wherein the at least one monovalent cation is lithium, potassium or sodium.

11. The composition of claim 9, wherein the divalent or trivalent cation is calcium, ferrous iron, ferric iron, magnesium, manganese, or zinc.

12. The composition of claim 1 wherein the inosine or ester or pharmaceutically acceptable salt and orotic acid, or salt thereof, is provided in one or more caplets, tablets, gelcaps, capsules or powders.

13. The composition of claim 12, wherein the pharmaceutical composition is in a solid dosage form.

14. A kit comprising in a first container a first pharmaceutical composition of orotic acid or acylating derivative or a salt thereof excluding a lysine salt thereof and a pharmaceutically acceptable carrier thereof and in a second container, a second pharmaceutical composition of inosine or ester thereof or salt thereof and a pharmaceutically acceptable carrier thereof, wherein inosine or ester or salt thereof and orotic acid or acylating derivative thereof or salt thereof are present in effective amounts for the treatment of a medical condition of a mammalian heart.

15. The kit according to claim 14, wherein the first and second pharmaceutical compositions are combined and administered in synergistically effective amounts.

16. The kit according to claim 14, wherein the first container is comprised of orotic acid or inorganic salt thereof.

17. The kit of claim 14, wherein the weight ratio of inosine or ester or a salt thereof to orotic acid or salt thereof or acylating derivative ranges from about 1:10 to about 10:1 .

18. The kit of claim 17, wherein the weight ratio of inosine or ester thereof or salt thereof to orotic acid or acylating derivative or salt thereof ranges from about 1:4 to about 4:1.

19. The kit of claim 14, wherein the calculated weight ratio of the inosine to the orotic acid ranges from about 1:10 to about 10:1.

20. The kit of claim 19, wherein the calculated weight ratio ranges from about 1:4 to about 4:1.

21. The kit of claim 14, wherein the inorganic salt thereof of orotic acid is selected from the group of orotate salts consisting of lithium, potassium, sodium, calcium, ferrous iron, ferric iron, magnesium, manganese, and zinc.

22. A method of treating cardiac disease in a subject in need thereof the method comprising, administering to the subject in need thereof, an amount effective to treat cardiac insufficiency of a combination of inosine or ester thereof or salt thereof and orotic acid or acylating derivative thereof or salt thereof.

23. The method of claim 22 wherein the inosine or salt thereof and the orotic acid or acylating derivative or salt thereof are administered in synergistically effective amounts.

24. The method of claim 22, wherein the weight ratio of inosine or salt thereof or ester thereof to orotic acid or acylating derivative or salt thereof the one or more inorganic salts thereof; is from about 1:10 to about 10:1.

25. The method of claim 22, wherein the weight ratio of inosine or salt or ester thereof to orotic acid or acylating derivative thereof or salt ranges from about 1:4 to about 4:1.

26. The method of claim 22, wherein the weight ratio of inosine or salt or ester to orotic acid or acylating derivative thereof or salt thereof is 1:4, 1:2, 2:3, 3:4, 1:1, 4:3, 3:2, 2:1 or 4:1.

27. The method of claim 22 wherein the cardiac insufficiency is caused by heart failure, myocardial infarction, arrhythmia, cardiomyopathy or myocardiodystrophy.

28. The method according to claim 22 wherein the cardiac disease is attributed to one or more of heart failure, myocardial infarction, arrhythmia, cardiomyopathy or myocardiodystrophy.

29. The method according to claim 22 wherein the cardiac disease is attributed to eccentric cardiac hypertrophy.

30. A method of increasing a subject's cardiac work capacity, the method comprising, administering to a subject, an effective amount of a combination of inosine or ester thereof, or salt thereof and orotic acid or acylating derivative thereof or salt thereof sufficient to enhance the work capacity performed by the subject.

31. The method of claim 30, wherein the wherein inosine or ester or salt thereof and orotic acid or acylating derivative thereof or salt thereof are present in synergistic amounts.

32. The method of claim 30, wherein the enhanced work capacity is either anaerobic or aerobic work, or a combination thereof.

33. A method of treating cardiac disease in a human subject, the method comprising administering to said human subject a composition comprising inosine or ester or salt thereof and orotic acid or acylating derivative thereof or a salt thereof in an amount that effectively improves the performance of the human subject's heart, wherein inosine or ester or salt thereof and orotic acid or acylating derivative thereof or salt thereof are present in effective amounts.

34. The method according to claim 33 wherein the inosine or ester or salt thereof and orotic acid r acylating derivative or salt thereof are present in synergistic effective amounts.

35. The method according to claim 33 wherein the cardiac disease is attributed to eccentric cardiac hypertrophy.
